(19) United States
(12) Patent Application Publication
(10) Pub. No.: US 2014/0065600 A1
(43) Pub. Date: Mar. 6, 2014

(54) METHOD OF EVALUATING ORAL CANCER RISK IN HUMAN

(71) Applicant: Institut Clinident, Saint Beauzire (FR)

(72) Inventor: Franck CHAUBRON, Venelles (FR)

(73) Assignee: Institut Clinident, Saint Beauzire (FR)

(21) Appl. No.: 13/975,256

(22) Filed: Aug. 23, 2013

Related U.S. Application Data
(62) Division of application No. 13/057,161, filed on Feb. 2, 2011.
(60) Provisional application No. 61/086,019, filed on Aug. 4, 2008.

(51) Int. Cl. C12Q 1/70

(52) U.S. Cl. CPC C12Q 1/708
USPC 435/5

(57) ABSTRACT

A method of providing a risk evaluation and diagnosis of human oral cancer, by examining at the presence in human saliva sample of a combination of particulate nucleic acids from bacteria, virus, as well as human, and/or the presence of particulate biochemical volatile organic compounds, which are indicative of an increased risk of oral cancer.

The present invention relates to a method of providing a risk evaluation and diagnosis of human oral cancer by examining, in a saliva sample of a human subject, the presence of particular nucleic acids of bacteria, virus, and/or human origin, as well as volatile biochemical organic compounds, a combination of which being indicative of an increased risk of oral cancer.

### BACKGROUND OF THE INVENTION

Cancers of the oral cavity accounted for 274,000 cases in 2002, with almost two-thirds of them in men. The world area with the highest incidence is Melanesia (31.5 per 100,000 in men and 20.2 per 100,000 in women). Rates in men are high in western Europe (11.3 per 100,000), southern Europe (9.2 per 100,000), south Asia (12.7 per 100,000), southern Africa (11.1 per 100,000), and Australia/New Zealand (10.2 per 100,000). In females, incidence of oral cancer is relatively high in southern Asia (8.3 per 100,000). These patterns reflect prevalence of specific risk factors, such as tobacco/alcohol, lack of dental and oral health and the chewing of betel quid in south central Asia and Melanesia. Moreover, for oral cavity cancer, the overall 5-year survival rates have not improved in the past several decades, remaining low at approximately 30-50% (Epstein, J. B. et al. 2002 J Can Dent Assoc 68: 617-621; Mao, L. et al. 2004 Cancer Cell 5: 311-316).

In addition, most of oral cancers are initially asymptomatic and are not diagnosed or treated until they reach an advanced stage. As of today, patients are questioned about associated risk to oral cancer (smoker, alcohol) followed by clinical inspection of oral cavity. Nonetheless, as indicated above early stages such as preneoplastic states and states of early tumor recurrence show no tissue damages that are visible by dentists or physicians.

Thus, there is a need for a method of assessing risk factor, for early diagnosis, and improved prognosis. In this regard, one problem is to provide a method that can be performed routinely and in the usual practice or laboratories.

The present invention disclosed a reliable and sensitive diagnostic method applied to the saliva of human subjects.

Saliva is a clear, slightly acidic fluid that contains a number of inorganic and organic constituents important to oral health. Whole saliva is a mix of secretions from major and minor salivary glands and gingival crevicular fluid, which contains sloughed host cells, bacteria and food debris. Therefore, saliva is not a passive “ultrafiltrate” of serum, but contains a distinctive composition of enzymes, hormones, antibodies, and other molecules (Rehak, N. N. et al. 2000 Clin Chem Lab Med 38:335-343; Wong D T, American Scientist, vol 96, 2008). For example, saliva contains a large number of proteins that aid in the protection of oral cavity tissues, including mucins, amylases, agglutinins, lisozymes, peroxidases, lactoferrin and secretory IgA. Whole saliva contains normal epithelial cells and leukocytes that can be pelleted, and from which one can easily recover genomic DNA and mRNA, potentially used to find genomic markers of several diseases. Indeed, most of the DNA or RNA extracted from crude saliva was found to be of viral or bacterial origin (Stamey, F. R. et al. 2003 J Virol Methods 108:189-193; Mercer, D. K. et al. 2001 FEMS Microbiol Lett 200:163-167) and of human extra or intracellular origin. Also, many groups have focused their study and diagnostic tests on the supernatant and thus cell-free phase of whole saliva, which contains many analytes such as free mRNA (Zimmermann B G et al, Oral Oncology 2008, 44, 425-429).

In the past 10 years, the use of saliva as been successfully applied in diagnostics (Streckfus, C F. & Bigler, L. R. 2002 Oral Dis 8:69-76). Diagnostic biomarkers in saliva have been identified for monitoring caries, periodontitis, salivary gland diseases, and systemic disorders, e.g., hepatitis and HIV (Lawrence H. P. et al, 2002 J Can Dent Assoc 68: 170-174). Also, oral bacteria have been reported to be elevated in oral and esophageal cancer lesions (Mager D. L. et al. J Transl Med. 2005; 3: 27, Hooper J. S et al., Journal of Clinical Microbiology, May 2006, P 1719-1725). The reason for these shifts in bacterial colonization of cancer lesions is unclear. Mechanistic studies of bacterial attachment provide some insights and research has repeatedly shown that oral bacteria demonstrate specific tropisms toward different biological surfaces in the oral cavity such as the teeth, mucosa, and other bacteria. There is less time in oral cavity, for a complex biofilm to develop on soft tissue surfaces; thus, a premium is placed on potent mechanisms of adhesion. The differences in bacterial tropisms for specific oral sites suggest that different intra-oral surfaces and bacterial species have different receptors and adhesion molecules that dictate the colonization of different oral surfaces. Certain glycoconjugates serve as receptors for specific bacteria and recent reports support the notion that shifts in the colonization of different cancer cells are associated with observed changes in cell surface receptors. Hence, Mager D. L. et al showed that the salivary microbiota in subjects with an oral squamous cell carcinoma (OSCC) lesion differs from that found in OSCC-free controls. Bacterial counts were determined for each species, averaged across subjects in the 2 subject groups, and significance of differences between groups determined using the Mann-Whitney test and adjusted for multiple comparisons; interestingly, it appeared that the bacteria strains Capnocytophaga gingivalis, Prevotella melaminogenica, Streptococcus mitis and Micrococcus luteus were particularly present in patients having OSCC and were therefore suggested to serve as diagnostic markers for oral cancer. However, as it is demonstrated in (ref), these particulate bacteria strains were poorly associated with oral cancer (a maximal sensitivity of 80%) (Mager D. L. et al). Also, it has been shown in Li et al (Journal of Applied Microbiology, 2004, 97, 1311-1318) that the presence in saliva of significant high numbers of specific alive bacteria (40 different strains have been identified in this study and more than 200 specific alive bacteria have been described in the oral cavity), could be associated to the biofilm formation, colonization of the oral cavity and lack of oral hygiene that are often associated to oral cancer development in developing countries. However, one can not predict from Li et al that the particulate strains Capnocytophaga gingivalis, Prevotella melaminogenica, Streptococcus mitis and Micrococcus luteus can serve as reliable diagnostic markers for human oral cancer. This is the reason why, to date, no reliable and very sensitive bacteria-based diagnostic test has ever been proposed to diagnose oral cancer in saliva.

It is interesting to note that the majority of species isolated were saccharolytic and acid tolerant and are known to produce short-chain organic acids from carbohydrates and consequently to lower the pH of their local environment. Raghunad N. et al. (J. Radiol. 2003; 76. S11-S22) described the microenvironment of solid tumors as is typically hypoxic, with an acidic extracellular pH so it is not surprising that there might be a degree of selectivity in favor of acid tolerance. Other factor could be production of DNA oxidative damage generated by oral bacteria. Takeuchi T. et al. (2000, FEMS Microbiol Lett. November 1; 192(1):133-8), investigated the mechanism of the oxidative DNA damage induction by exposure to O₂ in Prevotella melaminogenica, a strict anaerobe found in oral cavity and some dental diseases. Results indicate that in Prevotella melaminogenica, exposure to O₂ generated and accumulated O₂ and H₂O₂, and that a crypto-OH radical generated through H₂O₂ was the active species in the 8OHdG induction, highly responsible for oxidative DNA damage in human cells (genotoxic effect), possible cause of mammalian cell tumorigenicity in the oral environment. Other bacterial pathogens have been described as possible source of oral tissues cancerisation.

James J. Closmann, also found that oral and oropharyngeal squamous cell carcinoma (OOSCC) have been linked to high-risk HPV strains, the same strains that cause cervical cancer in women. D'Souza et al. (N Engl J. Med. 2007 May 10; 356(19):1944-56) concluded that oral HPV infection is strongly associated with oropharyngeal cancer among subjects with or without the established risk factors of tobacco and alcohol use. The degree of association increased with the number of vaginal-sex and oral-sex partners. Oropharyngeal cancer was associated with oral HPV type 16 (HPV-16) infection; oral infection with any of 37 types of HPV and seropositivity for the HPV-16 L1 capsid protein. HPV-16 DNA was detected in 72% of 100 paraffin-embedded tumor specimens, and 64% of patients with cancer were seropositive for the HPV-16 oncoprotein E6, E7, or both. HPV-16 L1 seropositivity was highly associated with oropharyngeal cancer among subjects with a history of heavy tobacco and alcohol use. The association was similarly increased among subjects with oral HPV-16 infection, regardless of their tobacco and alcohol use. Herrero R et al. (J Natl Cancer Inst. 2003 Dec. 3; 95(23):1772-83) conducted a multicenter case-control study of cancer of the oral cavity and oropharynx in nine countries with 1670 case patients (1415 with cancer of the oral cavity and 255 with cancer of the oropharynx) and 1732 control subjects HPV DNA was detected by polymerase chain reaction (PCR). Antibodies against HPV16 L1, E6, and E7 proteins in plasma were detected with enzyme-linked immunosorbent assays. Multivariable models were used for case-control and case-case comparisons. HPV DNA was detected in biopsy specimens of 3.9% of 766 cancers of the oral cavity with valid PCR results and 18.3% of 142 cancers of the oropharynx with valid PCR results. HPV16 DNA was found in 94.7% of HPV DNA-positive case patients. Co Consequently, it was suggested that antibodies against HPV16 L1 were associated with a high risk for cancers of the oral cavity and the oropharynx. Antibodies against HPV16 E6 or E7 were also associated with risk for cancers of the oral cavity and the oropharynx. HPV virus is even more of an indicator that patients should visit the dentist twice a year to identify anomalies early. Moreover, Rosenquist K. et al. (Acta Otolaryngol. 2007 September; 127(9):980-7), described that high-risk orally HPV-infected patients have a significantly higher risk of recurrence/second primary tumors compared with high-risk HPV-negative patients. Therefore, diagnosis of HPV in saliva of human subjects appears to be linked to a higher risk of developing oral cancer but also for monitoring associated treatment (antivirus) effects. However, to date, no HPV-based diagnostic test has ever been proposed to diagnose oral cancer in saliva.

On another hand, genetic aberrations of cancer cell lead to altered gene expression patterns (mRNA expression), which can be identified long before the resulting cancer phenotypes are manifested. Changes that arise exclusively or preferentially in cancer, compared with normal tissue of same origin, can be used as molecular biomarkers (Sidransky, D. 2002 Nat Rev Cancer 2:210-219, 2002). Accurately identified, biomarkers may provide new avenues and constitute major targets for cancer early detection and cancer risk assessment. A variety of nucleic acid-based biomarkers have been demonstrated as novel and powerful tools for the detection of cancers (Hollstein, M. et al. 1991 Science 253:49-53; Liu, T. et al. 2000 Genes Chromosomes Cancer 27:17-25; Groden, J. et al. 1991 Cell 66:589-600). These nucleic-acid based biomarkers are mostly studied in the cell-free phase of whole saliva samples, i.e. in filtrated or centrifugated saliva samples (Zimmermann B G et al, Oral Oncology 2008, 44, 425-429). However, there are only a limited number of reports demonstrating tumor cell DNA heterogeneity in saliva of oral cancer patients (Liao P. H. et al. 2000 Oral Oncol 36:272-276; El-Naggar, A. K. et al. 2001 J MolDiagn 3:164-170). Serum circulating human mRNA have been described for oral cancer detection (Yang Li et al, 2006, Journal of Clinical Oncology vol 24 number 11, 1754-1760). Using microarrays, profiling human salivary transcriptome (mRNA) have been realized on healthy controls and cancer patients (Hu S. and al.2006, J Dent Res. December; 85(12):1129-33). The different gene expression patterns were analyzed by combining a T test comparison and a fold-change analysis on 10 matched cancer patients and controls. The predictive power of these salivary mRNA biomarkers was analyzed by receiver operating characteristic curve and classification models. This microarray analysis showed that there are 1,679 genes exhibiting significantly different expression level in saliva between cancer patients and controls, among which 7 cancer-related the mRNA of IL8, IL1B, DUSP1, HA3, OAZ1, S100P, SAT are found. Combinations of these biomarkers yielded sensitivity (91%) and specificity (91%) in distinguishing oral cancer from the controls (WO2006/020005 or WO2005/081867).

Saliva is a mixture of secretions from multiple salivary glands, including the parotid, submandibular, sublingual and other minor glands lying beneath the oral mucosa. As mentioned before, human saliva harbors a wide spectrum of peptides and proteins that constitutes the human salivary proteome. What has been less studied is the presence of organic biochemical compounds in the saliva.

Biochemical organic compounds can be enzymes, hormones, inorganic ions, peptides, proteins, carbohydrates, vitamins, lipids, fatty acids and volatile compounds. They can be measured by many techniques and devices, either optical technologies (for example laser absorption spectroscopy, mid infra red absorption spectroscopy, laser magnetic resonance spectroscopy, proton transfer reaction mass spectrometry . . . ) or non-optical technologies (gas chromatography, mass spectrometry, etc. . . . ) (Mashir A, Advanced Powder Technology, 2009).

Only one study has ever compared the biochemical organic content of a fraction of saliva from healthy or sick-patients (Volozhin et al. Stomatologiia (mosk), 2001; 80(1):9-12). In this study, patients with chronic generalized periodontitis and patients with chronic generalized gingivitis and periodontitis have been tested with air from the oral cavity and liquid samples were collected by washing the oral cavity with sterile water. Chemical compounds of the air and the washed liquid were analyzed by chromato-mass-spectrometry, gas-adsorption and gas-liquid chromatography. The content of dimethyl sulphide, dimethyl disulphide increased in the oral air and such volatile short chain fatty acids (VSCFA) as butyrate, propionate, acetate rose, but their aldehydes (butyraldehyde, acrolein, acetaldehyde) decreased in oral fluid during periodontitis. It was also shown that volatile short-chain fatty acids (propionate, butyrate and acetate) of bacterial and tissue origin are important factors of pathogenesis of oral tissue inflammation (Volozhin et al. Stomatologiia (mosk), 2001; 80(1):9-12). In this study, the organic compounds have been analyzed in air from the oral cavity and rincing liquid collected by washing the oral cavity with sterile water but not in the volatile fraction of the raw saliva.

Contrary to saliva, the presence of volatile organic molecules in exhaled breath has been well studied and was shown to contain a lot of biochemical organic compounds: in 1971, using gas-liquid partition chromatography analysis, Linus Pauling demonstrated the presence of 250 substances in exhaled breath (Pauling L. et al. Proc.Natl.Acad.Sci.USA 68 (1971) 2374-2376). In 1990, the development of very sensitive modern mass spectrometry (MS) and gas chromatography mass spectrometry (GC-MS) instruments, gives identity to thousands of unique substances in human exhaled breath (Mashir A, Advanced Powder Technology, 2009). These substances include elemental gases like nitric oxide and carbon monoxide and a multitude of other volatile organic compounds. Furthermore, exhaled breath also carries aerosolized droplets collected as exhaled breath condensate that have non-volatile compounds that can be captured by a variety of methods and analyzed for a wide range of biomarkers from metabolic end products to proteins. Breath analysis is now used to diagnose and monitor asthma, pulmonary hypertension, respiratory diseases, gastrointestinal diseases, critical illness, to check for transplant organ rejection, and to detect lung cancer, and breast cancer (Mashir A, Advanced Powder Technology, 2009; Chan H. P. et al, Lung Cancer, 2009). However, it is noteworthy that breath analysis has never been proposed to detect oral cancer in human subject.

Interestingly, it appeared that the biochemical organic molecular composition of saliva has never been compared between patients suffering from cancer, e.g. oral cancer, and healthy individuals. Moreover, the biochemical organic molecular composition of the volatile fraction of saliva has never been studied so far.

The volatile fraction corresponds to the evaporated part of the fluid fraction of saliva. This volatile fraction contains some organic compounds that are not found in the raw saliva sample without the evaporation process, even after its filtration.

Also, it is important to note that the molecular content of saliva is not comparable at all to the composition of exhaled breath, which is mostly a reflect of lung molecules (Song G, et al. Quantitative breath analysis of volatile organic compounds of lung cancer patients, Lung Cancer (2009)). Hence, the molecular composition of the volatile fraction of saliva can not be inferred from the data issued from the breath analysis. To date, no exhaustive analysis of the biochemical content of the volatile fraction of saliva has never been disclosed, a fortiori in the context of oral cancer.

One of the values of saliva is the ease of sampling and subject compliance for sample collection, which includes field applications as well as home collection. However, the study of the biochemical compounds present in saliva (either in fluid or in the volatile fraction) for clinical application appeared to be difficult since it is necessary to stabilize and maintain their integrity for at least several days at room temperature.

It has already been shown that the RNAprotect® Saliva reagent (RPS, Qiagen Inc, Valencia, Calif.) could stabilize RNA in samples at room temperature for up to 12 weeks (Park N J, Clin Chem 2006; 52:2303-4). Also, Jiang J et al showed that it is possible to use the RPS for stabilization of DNA and proteins in saliva only when endogenous cells are previously removed by centrifugation or by filtration (Jiang J et al, Archives of Oral Biology, 2008).

As far as biochemical organic compounds are concerned, they are degraded by the microflora, food and dental care products at room temperature. Importantly, nobody has ever proposed a way to protect these sensitive compounds from the degradation occurring at room temperature. A fortiori, a buffer enabling the stabilization of both nucleic acids and biochemical organic compounds for several days at room temperature has never been described so far.

Therefore, it is still a major challenge to stabilize all the components of saliva, especially nucleic acids and organic compounds, without any filtration or centrifugation steps, for a long time at room temperature.

In this context, the present inventors show here for the first time that:
i) it is possible to stabilize the nucleic acids and organic compounds present in saliva during at least 10 days in an appropriate buffer,
ii) it is possible to detect a high risk of developing oral cancer by analyzing the level of only few particular biomarkers in the fluid fraction of a stabilized sample of saliva,
iii) it is possible to extract a volatile fraction from a sample of stabilized saliva, and to detect therein several organic volatile compounds in a significant amount,
iv) it is possible to detect a high risk of developing oral cancer by analyzing the level of only few particular biochemical compounds in said volatile fraction of stabilized saliva.

Importantly, it is to note that no test for predicting and/or diagnosing oral cancer using the volatile fraction of saliva has ever been proposed so far.

More importantly, the particular biochemical compounds hereafter identified have never been associated with cancer so far.

The present invention therefore discloses:
i) a method for stabilizing crude saliva samples of human subjects,
ii) a method for detecting a high risk of developing oral cancer, by analyzing, in a one-step reaction, the level of particular biomarkers in the fluid fraction of said stabilized saliva,
iii) a method for extracting the volatile fraction of stabilized saliva samples, and to analyze its content in biochemical organic compounds,
iv) a method for detecting a high risk of developing oral cancer, by analyzing the level of particular organic biochemical compounds in the volatile fraction of stabilized saliva.

Finally, specific combinations of biological parameters associated with high risk of oral cancer have been identified, highlighting that it is possible to obtain a reliable and sensitive prognosis/diagnosis test of oral cancer from a unique sample of stabilized saliva.

### FIGURE DESCRIPTION

FIG. 1 shows an histogram representing the 2 groups of saliva samples after a Factorial Discriminant Analysis (FDA) on ratios data according to the factor <<tumor/healthy>>: 5 ratios allow to separate 98.077% of the samples in the 2 groups <<tumor>> and <<reference group>> (only 1 reference sample is classified in the group <<tumor>> and not in the reference group).

### DESCRIPTION

The present invention disclose a new reliable, sensitive and easy to handle diagnostic test of oral cancer in human subject.

The present invention relates to an in vitro method of diagnosing a predisposition to oral cancer in a human subject, the method comprising stabilizing a crude saliva sample from said human subject, and performing at least one of the following steps a) and/or b):
a) Analyzing a fluid fraction of said stabilized saliva by detecting specific DNA or RNA sequences of human, bacterial or viral origin in said fluidic fraction,
b) Analyzing a volatile fraction extracted from said stabilized saliva by detecting in said volatile fraction at least one biochemical organic compound;
   wherein the detection of at least one DNA or RNA sequence as defined in a) and/or at least one biochemical organic compound as defined in b), is indicative of a risk or a predisposition to oral cancer.

The present invention enables to determine if a human subject is predisposed of developing an oral cancer or not.

When a human subject is “diagnosed to have a predisposition to oral cancer” or is found to be “predisposed to oral cancer”, it means that he has a higher risk of developing an oral cancer than the mean healthy population in the future. In other words, he is thought to be predisposed for developing an oral cancer in the early or far future. In the context of the invention, a human subject is said to be “predisposed to develop oral cancer” when he has a risk superior 70%, preferably 80%, more preferably 90% and even more preferably 95% of developing oral cancer in a short or far future as compared to a normal healthy population. This cancer predisposition is generally linked to a genetic cause.

The present invention also enables to determine that a human subject is “not predisposed to develop an oral cancer”. In this case, it means that he has a poor risk of developing oral cancer in the future. For example, it means that he has a risk of developing an oral cancer in the future lower than 10%, preferably lower than 5% as compared to the normal healthy population. It generally means that he has at least 90% of chance not to have oncogenic mutations in his genome.

The present invention also enables to diagnose an oral cancer in a human subject. As a matter of fact, the present invention disclosed an in vitro method of diagnosing an oral cancer in a human subject, comprising stabilizing a crude saliva sample from said human subject, and performing at least one of the following steps a) and/or b):
b) Analyzing a fluid fraction of said stabilized saliva by detecting specific DNA or RNA sequences of human, bacterial or viral origin in said fluidic fraction,
b) Analyzing a volatile fraction extracted from said stabilized saliva by detecting in said volatile fraction at least one biochemical organic compound;
   wherein the detection of at least one DNA or RNA sequence as defined in a) and/or at least one biochemical organic compound as defined in b), is indicative of a risk of developing oral cancer.

The method of the invention is thus dedicated to estimate a risk for a human subject of developing an oral cancer. This risk can be either a high risk of developing an oral cancer or a low risk of developing an oral cancer.

As used herein, when a human subject has a risk “of developing” an oral cancer, it means that he has a risk “to be developing” an oral cancer at the time of the collection of the saliva sample.

In the context of the invention, a human subject is said “to have a high risk of developing an oral cancer” when he has a risk at least superior to 60%, preferably to 70%, more preferably to 80% and even more preferably to 90% of developing an oral cancer. In other words, the human subject has a much higher probability to develop an oral cancer as compared to the normal population or to a human subject in which none of the organic compound is detected. In the context of the present invention, when a human subject has a risk superior to 97% to be developing an oral cancer, it is said that the human subject “is developing an oral cancer”.

This oral cancer can be initiating or well-established. In one embodiment of the invention, the level of expression of particulate biochemical organic compounds can potentially indicate the grade of the oral cancer from which the human subject is suffering.

The method of the invention also enables to determine if a human subject has a low risk to be developing an oral cancer. In the context of the invention, the human subject has a low risk of developing an oral cancer when he has a risk of developing an oral cancer lower than 10%, preferably lower than 5% as compared with the normal population. In other words, the human subject has a chance superior to 90%, preferably 95% to be healthy, at least as far as oral cancer is concerned. In the context of the invention, when a human subject has a risk inferior to 5% of being developing an oral cancer, it is said that the human subject is not developing an oral cancer at the time of the collection of the saliva sample.

In the context of the invention, the term “oral cancer” triggers the following cancers: cancer of the oral cavity, cancer of the oropharynx, oropharyngeal squamous cell carcinoma (OSCC), head and neck squamous cell carcinoma, preferably oral squamous cell carcinoma.

In the context of the invention, “collecting a crude saliva sample” is obtained by receiving, in a sterile device, a sample of the saliva that has been spitted by the human subject. A collecting reagent, for example a citrate buffer, may be added to the sample. This sample is then treated so as to stabilize it for later analysis, and to prepare it to nucleic acid and/or volatile fraction extraction.

It is one aspect of the present invention to provide a way to stabilize crude samples of saliva, so as to maintain high amounts of genetic markers and biochemical components as present in the initially spitted and collected saliva and to favour nucleic acid and/or biochemical volatile elements extraction. The present invention enables to maintain preferably at least 70%, more preferably 80% and even more preferably 90% of the amount of nucleic acids and biochemical organic components initially present in the spitted and collected saliva.

More precisely, the method of the invention enables to protect the raw components of the sample from degradation during at least 10 days at room temperature. This stabilization step is performed by adding to the crude sample a so-called “saliva preservation solution” comprising at least a preservation reagent, which is preferably a buffer comprising a salt capable of a) opening the membrane of bacteria and human cells, b) blocking nuclease activity, c) precipitating the nucleic acid and d) reducing the vapour tension of volatile compounds without allowing the degradation of said compounds. This salt is preferably a salt such as guanidinum thiocyanate, and/or ammonium sulfate and/or sodium azide, preferably sodium azide. This salt is employed preferably at a concentration range between 20 mM and 6 M, and more preferably at 40 mM.

The present invention therefore discloses a method for stabilizing the raw components of a crude saliva sample (such as nucleic acid and biochemical organic compounds) during at least 10 days at room temperature, said method comprising adding to the crude saliva sample a salt such as guanidinum thiocyanate, and/or ammonium sulfate and/or sodium azide. The salt is preferably sodium azide and is present at a concentration of about 40 mM.

Therefore, by “stabilized” saliva sample, it is meant to refer to samples in which nucleic acid species and organic volatile compounds have been preserved from degradation caused by the microflora, food and dental care products, during at least 10 days at room temperature.

In a first aspect, the present invention provides a method of diagnosing a risk or a predisposition to oral cancer in a human subject, simply by using a stabilized sample of its saliva and analyzing its fluid fraction by detecting specific DNA or RNA sequences of human, bacterial or viral origin.

As used herein, the “fluid fraction” (or “fluidic fraction”) of a saliva sample is the liquid phase of the collected saliva, and contains cells as well as free nucleic acids and organic compounds.

In the method of the invention, and contrary to what has been described so far, there is no need to remove the cells from the crude saliva samples to obtain the fluid fraction. Therefore, the saliva fluid samples are preferably not centrifugated and not filtrated.

As used herein, the expression “DNA or RNA sequences” refer to total nucleic acid in the sample and comprises genomic DNA and total RNA including mRNA.

Preferably, said specific DNA or RNA sequences are chosen among:
i) human sequences selected from SSAT mRNA (SEQ ID No 62), H3F3A mRNA (SEQ ID No 63) and IL8 mRNA (SEQ ID No 64); and/or
ii) sequences of bacteria selected from Capnocytophaga gingivalis (ATCC 33624), Prevotella melaminogenica (ATCC 25845), Streptococcus mitis (ATCC 15914) and Micrococcus luteus (ATCC 53598D); and/or
iii) the viral sequences of human papillomavirus, preferably the human papillomavirus 16 (ATCC 45113) or the human papillomavirus 18 (ATCC 45152).

Preferably, specific DNA or RNA sequences (also called nucleic acid sequences) are detected by incubating said genomic DNA and total RNA with a thermostable enzyme with RNA-dependent Reverse Transcriptase activity and with DNA-dependent Polymerase activity.

More preferably, the combination of RT and PCR is performed in a single-tube reaction.

The term “detecting” as used herein is meant to refer to diagnosing, inferring, evaluating, monitoring, determining the amount, concentration, ratio, or other quantitative or qualitative assessment in samples, optionally compared to a control sample, of nucleic acid and volatile compounds.

In the context of the present invention, “detecting specific nucleic acid sequences” comprises comparing the expression level of the specific nucleic acid sequences to the mean expression level of said specific nucleic acid sequences in the normal population. A specific nucleic acid sequence is detected when the expression level of said specific nucleic acid sequences in the tested saliva sample is equivalent or superior to 2 fold the mean expression level of said specific nucleic acid sequences in the normal population.

The method of the invention may comprise comparing the level of expression of specific nucleic acid sequence in a saliva sample from a subject, with the normal expression level of said nucleic acid sequence in a control. A significantly higher level of expression of said gene in the saliva sample of a subject as compared to the normal expression level is an indication that the patient has or is predisposed to oral cancer. An “over-expression” of a specific nucleic acid refers to an expression level in a saliva sample that is greater than the standard error of the assay employed to assess expression, and is preferably at least 20% superior to the normal level of expression of said nucleic acid, preferably at least 50% superior to the normal level of expression of said nucleic acid, and most preferably at least 100% superior to the normal level of expression of said nucleic acid. The “normal” level of expression of a nucleic acid is the level of expression of said nucleic acid in a saliva sample of a subject not afflicted with cancer. Preferably, said normal level of expression is assessed in a control sample (e.g., sample from a healthy subject, which is not afflicted by cancer) and preferably, the mean expression level of said nucleic acid in several control samples.

Also, the detection of specific nucleic acid sequences is based on the detection of at least 50, preferably 70, and even more preferably 100 successive nucleotides of the specific targeted nucleic acid sequence as registered in the official data bases, for example in the NCBI data base.

For nucleic acid sequences according to ii) as defined above, “detecting” comprises comparing the expression level to the expression level in the normal population or to mean expression level and wherein when expression level is equivalent or superior of the threshold/cutoff of 10⁵ CFU per ml of saliva, it is indicative of increased oral cancer.

In one embodiment of the present invention, the detection of at least two human mRNA sequences as specifically defined in i) and at least one bacterial sequence as specifically defined in ii) indicates that the human subject has a high risk of developing an oral cancer.

In a preferred embodiment of the present invention, the detection of at least the human mRNA of H3F3A (SEQ ID No 63) and the human mRNA of SSAT (SEQ ID No 62) in the saliva sample of a human subject indicates that said human subject has a high risk developing an oral cancer.

In a more preferred embodiment of the present invention, the detection of the human mRNA of H3F3A (SEQ ID No 63), the human mRNA of SSAT (SEQ ID No 62) and the bacterial genome of Streptococcus mitis (ATCC 15914) in the saliva sample of a human subject indicates that said human subject has a risk superior to 64% of developing an oral cancer, and has therefore a high risk of developing an oral cancer.

On the contrary, when the particulate DNA or RNA sequence as disclosed in the present invention are not detected in the saliva sample of a human subject, it means that said human subject has a low risk of developing an oral cancer.

As mentioned previously, one problem was to design a test detecting the various risk factors actually associated with oral cancer in one simple test reaction. Indeed, some important biological markers are of different origins, virus, bacteria, human, or from mRNA or DNA, which makes the detection difficult in one step reaction test.

In one embodiment of the present invention, the detection of nucleic acid sequences comprises incubating said genomic DNA and total RNA with a thermostable enzyme with RNA-dependent Reverse Transcriptase activity and with DNA-dependent Polymerase activity, allowing the combination of RT-PCR and PCR.

Preferably, the RT-PCR reaction is performed with the Tth DNA polymerase.

More preferably, the detection of nucleic acid sequences comprises incubating said genomic DNA and total RNA in the same tube with a thermostable enzyme with RNA-dependent Reverse Transcriptase activity and with DNA-dependent Polymerase activity, allowing the combination of RT-PCR and PCR in a single-tube reaction.

Even more preferably, the detection of nucleic acid sequences comprises amplifying and detecting at least one DNA or RNA sequence chosen among SEQ ID No 62 to 70.

Therefore, in a preferred embodiment, the present invention is drawn to a method for diagnosing a predisposition to oral cancer, or for diagnosing an oral cancer in a human subject, the method comprising the steps of
a) extracting total nucleic acid (genomic DNA and total RNA) of bacteria, virus, and human origins from the stabilized saliva sample,
b) incubating said genomic DNA and total RNA in the same tube with a thermostable enzyme with RNA-dependent Reverse Transcriptase activity and with DNA-dependent Polymerase activity, allowing the combination of RT and PCR in a single-tube reaction,
   wherein said RT-PCR reaction is performed with primers and probes specific to:
i) human sequences selected from SSAT mRNA (SEQ ID No 62), H3F3A mRNA (SEQ ID No 63) and IL8 mRNA (SEQ ID No 64);
ii) sequences of bacteria selected from Capnocytophaga gingivalis (ATCC 33624, SEQ ID No 65), Prevotella melaminogenica (ATCC 25845, SEQ ID No 66), Streptococcus mitis (ATCC 15914, SEQ ID No 67) and Micrococcus luteus (ATCC 53598D, SEQ ID No 68),
iii) sequences of virus selected from human papillomavirus 16 (ATCC 45113, SEQ ID No 69) and human papillomavirus 18 (ATCC 45152, SEQ ID No 70);
   and wherein the presence of at least one sequence of each i), ii) or iii), is indicative of a risk or a predisposition to oral cancer.

In a particular embodiment, the presence of at least one sequence of each i), ii) and iii), is indicative of a risk or a predisposition to oral cancer.

More specifically, the detection of the presence of a combination of at least one of said sequences iii), at least two of said sequences i) and at least two of said sequences ii) indicates that the human subject has an increased risk of developing oral cancer.

In one particular embodiment, the method comprises amplifying and detecting SEQ ID No 62 to 70.

In another particular embodiment, the invention is directed to a method for risk evaluation and diagnosis of oral cancer disease in a human subject comprising the steps of:

a) collecting a sample of crude saliva of said human subject in a sterile device,
b) stabilizing said sample by adding a solution comprising a guanidium salt, such as guanidinum thiocyanate, and/or ammonium sulfate, and/or sodium azide, and optionally exo and/or endonuclease inhibitors,
c) extracting total nucleic acid of bacteria, virus, and human origins from the previously obtained stabilized saliva sample,
d) precipitation and purification of total nucleic acids,
e) incubating the purified total nucleic acid with a thermostable enzyme with RNA-dependant reverse transcriptase activity and with DNA-dependant polymerase activity and polynucleotide primers under conditions which allow the reverse transcriptase activity of said thermostable enzyme to synthetise cDNA from the ribonucleic and amplification of genomic DNA and cDNA at a detectable level by Polymerase Chain Reaction,
f) detecting in an assay the amplified DNAs sequences by hybridization with one or more polynucleotide probes specific to:
i) human sequences selected from SSAT mRNA (SEQ ID No 62), H3F3A mRNA (SEQ ID No 63) and IL8 mRNA (SEQ ID No 64); or
ii) sequences of bacteria selected from Capnocytophaga gingivalis (ATCC 33624, SEQ ID No 65), Prevotella melaminogenica (ATCC 25845, SEQ ID No 66), Streptococcus mitis (ATCC 15914, SEQ ID No 67) and Micrococcus luteus (ATCC 53598D, SEQ ID No 68), or
iii) sequences of virus selected from human papillomavirus 16 (ATCC 45113, SEQ ID No 69) and human papillomavirus 18 (ATCC 45152, SEQ ID No 70).

Preferably, said method is performed by detecting the amplified DNAs sequences by hybridization with one or more polynucleotide probes specific to i), ii) and iii).

In the above method, it is also contemplated to add a calibrated and know nucleic acid (DNA or RNA) to the vial used to collect saliva to measure the exact quantity of saliva collected by comparison with an external standard nucleic acid calibration curve.

Regarding step d), total nucleic acid present in the preservative reagent can be purified by a nucleic acid affinity resin.

In step e), the purified genomic DNA and total RNA are incubated in the same tube with a thermostable enzyme with RNA-dependent Reverse Transcriptase activity and with DNA-dependent Polymerase activity, allowing the combination of RT and PCR in a single-tube reaction, such as Tth DNA polymerase, and polynucleotide primers with nucleotides sequences that detect at least one of each i), ii) or iii) sequences.

According to the invention, polynucleotide primers and probes are natural nucleic acid or Peptide Nucleic Acid (PNA) or modified nucleic acid (superbase) which can hybridize to nucleic acid (DNA and RNA).

Step f) may further comprise the step of quantifying amplified DNA by comparison with quantified standard DNA or RNA and determining whether or not the nucleic acid is present, over present or overexpressed in the saliva sample.

In one particular embodiment, the invention relates to a method of measuring the presence of oral cancer risk factors in a human subject comprising the steps of:
The recovery and preservation of total nucleic acid from crude saliva from degradation caused by nucleases,
Optionally, the addition of a positive nucleic acid control for the full analytic process and exact quantification of the saliva amount,
Nucleic acid extraction and concentration of total nucleic acid (genomic DNA and total RNA),
A one-step Reverse Transcriptase Polymerase Chain Reaction: The purified genomic DNA and total RNA are incubated in the same tube with a thermostable enzyme with RNA-dependent Reverse Transcriptase activity and with DNA-dependent Polymerase activity, allowing the combination of RT and PCR in a single-tube reaction, such as Tth DNA polymerase, and polynucleotide primers with nucleotides sequences that detect Capnocytophaga gingivalis (ATCC 33624, Genbank Accession AF543295), Prevotella melaminogenica (ATCC 25845, Genbank Accession N^{o} AJ555137), Streptococcus mitis (ATCC 15914, Genbank Accession N^{o} AJ617805,) Micrococcus luteus (ATCC 53598D, Genbank Accession N^{o} AM285006), human papillomavirus 16 (ATCC 45113, Genbank Accession N^{o} EF422141), human papillomavirus 18 (ATCC 45152, Genbank Accession N^{o} EF422111), SSAT mRNA (Genbank Accession N^{o} NM002970), H3F3A mRNA (Genbank Accession N^{o} NM002107), IL8 mRNA (Genbank Accession N^{o} NM000584).

Using one-step Real-time Reverse Transcriptase Polymerase Chain Reaction, the invention enables the user to perform a rapid RT-PCR and simultaneously detect and quantify the presence of total nucleic acid from bacteria, virus and human mRNA by monitoring fluorescence during real time polymerase chain reaction amplification without any risk of false positive due to opening tube between RT and PCR and from possible PCR product environmental contamination due to precedent amplification reactions in the same environment.

Beta actine mRNA (Genbank Accession Number X00351—SEQ ID No 61) is useful as internal standard in step a) for calibration and quantification.

In a preferred embodiment, the polymerase with RNA-dependent Reverse Transcriptase activity and with DNA-dependent Polymerase activity is the Tth DNA polymerase.

Using total nucleic acid extraction and one-step Real-time Reverse Transcriptase Polymerase Chain Reaction in a same microfluidic cartridge, the invention enables the user to perform a point of care analysis usable in physician and dentist offices.

Alternatively, using one-step Reverse Transcriptase Polymerase Chain Reaction and a microarray, the invention enables the user to perform RT-PCR and detect and quantify the presence of total nucleic acid from bacteria, virus and human mRNA by measuring hybridization signal at the end of the RT-PCR and compare with external nucleic standard.

In a specific embodiment, the present invention is drawn to a micro array for diagnosing a predisposition to oral cancer in a human subject comprising probes sequences selected from the group consisting of SEQ ID No 32 to SEQ ID No 60.

In this aspect of the present invention, the polynucleotide primers and probes to amplify and detect at least one of each i), ii) or iii) sequences are preferably selected from the group consisting of SEQ ID No:1 to SEQ ID No:60.

Kit of parts associated with the methods herein disclosed are also disclosed. In an exemplary embodiment, the present invention therefore relates to a kit to practice the above method, comprising primers and probes sequences to amplify and detect at least one of each of the sequences i), ii) or iii) depicted above, and including at least one of the following:

a) a sterile device to collect a saliva sample, optionally including a control nucleic acid, and a collect reagent,
b) a preservation reagent, for example a spray dry preservative reagent,
c) a resin having affinity for total nucleic acid,
d) a thermostable enzyme with RNA-dependant reverse transcriptase activity and with DNA-dependant polymerase activity and polynucleotide.

As mentioned before, the collect reagent is a dilution buffer which is preferably citrate buffer.

The kit comprises a preservation reagent, which is preferably a buffer comprising a salt such as guanidinum thiocyanate, and/or ammonium sulfate and/or sodium azide, and optionally exo and/or endonuclease inhibitors. The sodium azide salt is employed preferably at a concentration range between 20 mM and 100 mM, more preferably around 40 mM. Other salts, such as guanidium thiocyanate and/or ammonium sulphate, are added at a concentration of 4M.

In one embodiment of the present invention, the preservation reagent is provided in dry format in a sterile plastic tube under vacuum, which can draw up the crude saliva associated with the dilution buffer.

Primers and probes sequences to amplify and detect at least one of each of the sequences i), ii) or iii) depicted above are further defined as comprising polynucleotide primers for synthesizing cDNA by reverse transcription, polynucleotide primers for amplifying genomic DNA and cDNA by polymerase chain reaction and polynucleotide probes for detecting amplified DNA.

In the diagnostic kits herein disclosed, the reagent can be provided in the kits, with suitable instructions and other necessary reagents in order to perform the methods here disclosed. Instructions, for example written or audio instructions, on paper or electronic support such as tapes or CD-roms, for carrying out the assay, will be usually included in the kit.

In a second aspect, the present invention is drawn to a method of diagnosing a predisposition to oral cancer in a human subject, the method comprising collecting and stabilizing a crude saliva sample from said human subject, and analyzing a volatile fraction extracted from said stabilized saliva by detecting in said volatile fraction at least one biochemical organic compound, wherein the detection of at least one biochemical organic compound is indicative of a risk or a predisposition to oral cancer.

The present invention is also drawn to an in vitro method of diagnosing oral cancer in a human subject, the method comprising stabilizing a crude saliva sample from said human subject, and analyzing a volatile fraction extracted from said stabilized saliva by detecting in said volatile fraction at least one biochemical organic compound, wherein the detection of at least one biochemical organic compound is indicative of a risk of developing an oral cancer.

From a chemistry point of view, biochemical organic compounds are the members of a large class of chemical compounds whose molecules contain carbon. They can be antigens, carbohydrates, enzymes, hormones, lipids, fatty acids, neurotransmitters, nucleic acids, proteins, peptides and amino acids, vitamins, fats and oils.

Among all the known organic compounds, “Volatile organic compounds” (VOC) are meant to designate any organic compound that is volatile, i.e. that have a high vapor pressure or low boiling point, and can therefore evaporate at normal temperature and pressure. These compounds are often regulated by governments. For example, in European Union, a “Volatile Organic Compound” is any organic compound having an initial boiling point less than or equal to 250° C. measured at a standard atmospheric pressure of 101.3 kPa.

In the context of the invention, the “volatile fraction” is recovered from the heating of a crude saliva sample. Preferably, said volatile fraction is extracted from crude saliva sample by heating said saliva sample for at least 10 minutes, preferably 20 minutes and more preferably 30 minutes at a temperature comprised between 30° C. and 50° C., preferably 40° C. During this time, the volatile fraction is taken away from the sample by using a solid-phase microextraction (SPME) with a carboxen/polydimethylsiloxane coated fiber (CAR/PDMS fiber).

Therefore, in one embodiment of the present invention, the volatile biochemical organic compounds are extracted with a CAR/PDMS fiber coating during at least, preferably 20 minutes, and even more preferably 30 minutes from a saliva sample that is simultaneously heated at a temperature comprised between 30° C. and 50° C., and preferably at about 40° C. The desorption temperature of the fiber is comprised between 250° C. and 300° C., and is preferably of about 280° C.

Solid-phase microextraction (SPME) is a patented sample preparation technique based on the adsorption of analytes directly from an aqueous sample onto a coated, fused-silica fiber. This sampling technique is fast, easy to use and eliminates the use of organic solvents (Mills G et al, Journal of Chromatography 2000; Song C et al, Lung Cancer 2009).

In this technology, the CAR/PDMS fibers are often used for detecting trace level of volatile compounds, and are therefore well-known from the man skilled in the art (Garcia-Esteban M et al, Talanta 2004).

Preferably, the detection of said biochemical organic compound is performed by using a chromatograph in gas phase coupled to a mass spectrometer.

In the context of the invention, a biochemical compound is “detected” when the expression level of said compound is at least superior to 1.5 fold the mean expression level of said compound in the normal population.

By applying the method of the invention, some biochemical compounds were shown to be highly overexpressed in the volatile fraction of human subjects suffering from oral cancer and were therefore found to be acute and sensitive diagnostic and/or prognostic tool of oral cancer. Importantly, none of these compounds can be detected in the fluid fraction of saliva, highlighting the necessity to study the volatile fraction of saliva in this case.

In a preferred embodiment, the present invention is drawn to a method of diagnosing a risk or a predisposition to oral cancer in a human subject by analyzing the content in biochemical compounds in the volatile fraction of stabilized samples of saliva, wherein the detection of at least one of the biochemical organic compound selected in the group consisting of: 2,3-pentanedione (CAS number 600-14-6), 3-methyltiophene (CAS number 616-44-4), acetone (CAS number 67-64-1), hexanenitrile (CAS number 628-73-9), benzaldehyde (CAS number 100-52-7), 3-methyl-2-pentanone (CAS number 565-61-7), 2,3-butanedione (CAS number 431-03-8), 2-propanol (CAS number 67-63-0), ethyl acetate (CAS number 141-78-6), 1-propanol (CAS number 71-23-8), hexanal (CAS number 66-25-1), 5-methyl-3-hexen-2-one (CAS number 5166-53-0), m-xylene (CAS number 108-38-3), p-xylene (CAS number 106-42-3), 2-methyl-2-butenal (E) (CAS number 497-03-0), phenol (CAS number 108-95-2), butanal (CAS number 123-72-8), methylbutanone (CAS number: 563-80-4), 2-methyl-2-butene (CAS number 513-35-9), 2-methyl-1-propene (CAS number 115-11-7) and (cis) 1,2 dimethyl-cyclopropane (CAS number: 930-18-7) is indicative of a risk or predisposition to oral cancer.

Indeed, the below-presented results have shown that the detection of at least one of the following compounds: 2,3-pentanedione (CAS number 600-14-6), 3-methyltiophene (CAS number 616-44-4), acetone (CAS number 67-64-1), hexanenitrile (CAS number 628-73-9), benzaldehyde (CAS number 100-52-7), 3-methyl-2-pentanone (CAS number 565-61-7), 2,3-butanedione (CAS number 431-03-8), 2-propanol (CAS number 67-63-0), ethyl acetate (CAS number 141-78-6), 1-propanol (CAS number 71-23-8), hexanal (CAS number 66-25-1), 5-methyl-3-hexen-2-one (CAS number 5166-53-0), m-xylene (CAS number 108-38-3), p-xylene (CAS number 106-42-3), 2-methyl-2-butenal (E) (CAS number 497-03-0) in the volatile fraction of saliva of a human subject indicates that said human subject has a high risk of being predisposed to develop oral cancer or has a high risk of being developing an oral cancer. More precisely, the detection of at least one of the above-mentioned biochemical organic compounds indicates that the tested human subject has a risk superior to 60% of developing an oral cancer; the detection of at least two of the above-mentioned biochemical organic compounds indicates that the tested human subject has a risk superior to 70% of developing an oral cancer, and the detection of at least three of the above-mentioned biochemical organic compounds indicates that the tested human subject has a risk superior to 80% of developing an oral cancer; the detection of at least four of the above-mentioned biochemical organic compounds indicates that the tested human subject has a risk superior to 90% of developing an oral cancer. On the contrary, when at least one of the particulate biochemical organic compounds disclosed above is not detected in the saliva sample of a human subject, it means that said human subject has a low risk of developing an oral cancer.

For example, as shown in the example 10, the detection of the biochemical organic compounds of the group comprising: hexanitrile, 2,3-pentanedione, 3-methylthiophene and acetone in the volatile fraction of saliva of a human subject indicates that said human subject has a risk superior to 97% of developing an oral cancer, and is therefore predisposed to develop an oral cancer, or is developing an oral cancer.

Also, as shown in the example 11, the detection of the mRNA sequence of H3F3A (SEQ ID No 63), SSAT (SEQ ID No 62) and of the biochemical organic compounds hexanenitrile, 2,3-pentanedione, 3-methylthiophene and acetone in the saliva of a human subject enables to detect 100% of cancer cases in a tested population. Therefore, in a preferred embodiment, the detection of the mRNA sequence of H3F3A (SEQ ID No 63), SSAT (SEQ ID No 62) and of the biochemical organic compounds hexanenitrile, 2,3-pentanedione, 3-methylthiophene and acetone in the saliva of a human subject indicates that said human subject is developing an oral cancer.

Also, as exemplified hereunder, the method of the present invention can rely on the detection of 3-methyl-2-pentanone, methyl butanone, butanal, hexanal, hexanenitrile, 1-propanol 2-propanol, (cis) 1,2-dimethyl cyclopropane, phenol, and 2,3-butanedione in order to prognose or to diagnose an oral cancer in a human subject.

In another embodiment, the present invention is drawn to a method of diagnosing a risk or a predisposition to oral cancer in a human subject, wherein the detection of at least one biochemical organic compound in the volatile fraction of saliva of a human subject indicates that said human subject is not predisposed to develop an oral cancer or is not developing an oral cancer.

The compounds 2-methyl-2-butene (CAS number 513-35-9), 2-methyl-1-propene (CAS number 115-11-7) and (cis) 1,2-dimethyl cyclopropane (CAS number 930-18-7) are overexpressed in the volatile fraction of healthy human subject and are absent in the volatile fraction of patients suffering from oral cancer. Therefore, these compounds can serve as “healthy biochemical markers”. The detection of at least one, preferably two of these biochemical organic compounds indeed indicates that the human subject has a low risk of being predisposed of being developing an oral cancer.

Moreover, the detection of particulate biochemical organic compounds such as benzaldehyde, acetone, 2,3-pentanedione, on a one hand, and the absence of other particulate biochemical compounds such as 2-methyl-2-butene, 2-methyl-1-propene, and/or (cis) 1,2-dimethyl cyclopropane on the other hand, enables to diagnose oral cancer with a high sensitivity (at least 93%, see example 10).

It is noteworthy that most of these biochemical compounds have never been identified so far in the saliva. Moreover, none of them have been related so far with cancer predisposition, and a fortiori with oral cancer predisposition.

Therefore, the present invention is also drawn to the use of the detection of at least one compound chosen among: 2,3-pentanedione (CAS number 600-14-6), 3-methyltiophene (CAS number 616-44-4), acetone (CAS number 67-64-1), hexanenitrile (CAS number 628-73-9), benzaldehyde (CAS number 100-52-7), 3-methyl-2-pentanone (CAS number 565-61-7), 2,3-butanedione (CAS number 431-03-8), 2-propanol (CAS number 67-63-0), ethyl acetate (CAS number 141-78-6), 1-propanol (CAS number 71-23-8), hexanal (CAS number 66-25-1), 5-methyl-3-hexen-2-one (CAS number 5166-53-0), m-xylene (CAS number 108-38-3), p-xylene (CAS number 106-42-3), 2-methyl-2-butenal (E) (CAS number 497-03-0), 2-methyl-2-butene (CAS number 513-35-9), 2-methyl-1-propene (CAS number 115-11-7) and (cis) 1,2-dimethyl cyclopropane (CAS number 930-18-7) in a diagnostic test to assess the risk of developing an oral cancer in a human subject.

In a particulate embodiment, the present invention is therefore drawn to a method of diagnosing a risk or a predisposition to oral cancer in a human subject, comprising the steps of:
a) collecting a sample of crude saliva of said human subject in a sterile device,
b) stabilizing said sample by adding a solution comprising a salt, such as guanidinum thiocyanate, ammonium sulfate and/or sodium azide,
c) extracting the volatile fraction from said stabilized sample by heating it for at least 10 minutes at 40° C. and using for example Solid-phase Microextraction (SPME) to take away the volatile fraction,
d) detecting at least one biochemical organic compound by using for example a chromatograph in gas phase coupled to a mass spectrometer,
   wherein the detection of at least one, preferably at least two, and more preferably at least three biochemical organic compound(s) is indicative of a risk or a predisposition to oral cancer.

In a preferred embodiment, the at least one, preferably at least two, and more preferably at least three detected biochemical organic compound is (are) chosen among: 2,3-pentanedione (CAS number 600-14-6), 3-methyltiophene (CAS number 616-44-4), acetone (CAS number 67-64-1), hexanenitrile (CAS number 628-73-9), benzaldehyde (CAS number 100-52-7), 3-methyl-2-pentanone (CAS number 565-61-7), 2,3-butanedione (CAS number 431-03-8), 2-propanol (CAS number 67-63-0), ethyl acetate (CAS number 141-78-6), 1-propanol (CAS number 71-23-8), hexanal (CAS number 66-25-1), 5-methyl-3-hexen-2-one (CAS number 5166-53-0), m-xylene (CAS number 108-38-3), p-xylene (CAS number 106-42-3) and, 2-methyl-2-butenal (E) (CAS number 497-03-0), phenol (CAS number 108-95-2), butanal (CAS number 123-72-8), methylbutanone (CAS number: 563-80-4), 2-methyl-2-butene (CAS number 513-35-9), 2-methyl-1-propene (CAS number 115-11-7) and (cis) 1,2 dimethyl-cyclopropane (CAS number: 930-18-7).

In a preferred embodiment, the detection of the at least one detected biochemical compound chosen among 2,3-pentanedione (CAS number 600-14-6), 3-methyltiophene (CAS number 616-44-4), acetone (CAS number 67-64-1), hexanenitrile (CAS number 628-73-9), benzaldehyde (CAS number 100-52-7), 3-methyl-2-pentanone (CAS number 565-61-7), 2,3-butanedione (CAS number 431-03-8), 2-propanol (CAS number 67-63-0), ethyl acetate (CAS number 141-78-6), 1-propanol (CAS number 71-23-8), hexanal (CAS number 66-25-1), 5-methyl-3-hexen-2-one (CAS number 5166-53-0), m-xylene (CAS number 108-38-3), p-xylene (CAS number 106-42-3), and 2-methyl-2-butenal (E) (CAS number 497-03-0) is indicative of a high risk of developing cancer as compared to a normal healthy population.

On the contrary, when at least one of the biochemical compounds: 2-methyl-2-butene (CAS number 513-35-9), 2-methyl-1-propene (CAS number 115-11-7) or (cis) 1,2-dimethyl cyclopropane (CAS number 930-18-7) is detected, it is indicative of a poor risk of developing oral cancer, i.e. a risk inferior to 10%, preferably 5% to develop cancer as compared to a normal healthy population.

In another embodiment, the present invention is drawn to a kit to practice a method of diagnosing a risk or a predisposition to oral cancer based on the volatile fraction of saliva, comprising:
a) A sterile device to collect a saliva sample, optionally containing a collect reagent
b) A preservation reagent,
c) At least one electronic sensor,
d) Optionally, a control molecular marker.

As mentioned before, the collect reagent is a dilution buffer which is preferably a citrate buffer.

The kit comprises a preservation reagent, which is preferably a buffer comprising a salt capable of reducing the vapour tension of volatile compounds without allowing the degradation of said compounds. This salt is preferably a salt such as guanidinum thiocyanate, and/or ammonium sulfate and/or sodium azide. This sodium azide is employed preferably at a concentration range between 20 mM and 6M, preferably between around 10 mM and 100 mM, more preferably around 40 mM. Other salts, such as guanidium thiocyanate and/or ammonium sulphate, are added at a concentration of 4M.

In one embodiment of the present invention, the preservation reagent is provided in a dry format in a sterile plastic tube under vacuum, which can draw up the saliva associated with the dilution buffer.

In the context of the invention, the device used to detect the organic compounds in the collected volatile fraction of saliva is an electronic sensor, for example electronic noses, JPL electronic noses, FET-type Bioelectronic noses, alpha mos). These technologies are now widely used and therefore known from the man skilled in the art (Cho S. M., Sensors and Actuators 2006).

Using specific electronic sensors for the identification of the targeted volatile compounds in a specific platform (electronic nose), the invention enables the user to perform a specific analysis platform or a point of care analysis usable in physician and dentists offices.

In one embodiment of the invention the control molecular markers are chosen among: 1-bromobutane, 1-bromobenzene and 1,4-dibromobenzene.

### Example 1

### Stabilization of Crude Samples of Saliva

Raw saliva is collected with a medical device which makes easier the collection of a large volume of saliva (up to 2 ml) following by a stabilization of the saliva biomarkers (AND, ARN, peptides, volatile compound).

4 ml of saliva extraction solution is then swallowed up to 2 minutes for collection of 2 ml of saliva.

The saliva extraction solution contains:
FD&C yellow n^{o} 5 (tartrazine)
Citrate buffer (39 mM)

The 2 ml of diluted saliva (in 4 ml) are then transferred in tubes containing lyophilized sodium azide for biomarker stabilization. The final sodium azide concentration is about 40 mM.

2 tubes are prepared for each sample. A tube is intended for the “genetic” analyzes (tube 1) and the other tube (tube 2) is used for the analyzis of the volatile compounds.

The tubes can be transported without control of temperature during 10 days before being analyzed.

### Example 2

### Simultaneous Determination and Quantification in Stabilized Saliva of the Presence of Bacteria, Virus and Human mRNA to Assess Risk Factor of Oral Cancer

0.250 to 3 ml of the solution from tube 1 is prepared and total nucleic acids are extracted by centrifiltration on a silica membrane without DNAse step. Up to 1.5 mg of total nucleic acid are extracted and purified from 1 ml of stabilized saliva.

Isolation of high-quality DNA and RNA from whole saliva samples is difficult because under ambient conditions, expression and quantification profile are unstable on a timescale below few minutes. This instability is the result of metabolic activity of bacteria, nutrients dependant, concentration in nucleases and limited turnover of RNA in that environmental conditions. In order to render the nucleic acid inaccessible to nuclease, we used a preservation buffer which comprises a salt for membrane lysis of bacteria as well as human cells and precipitates the extracted nucleic acid in the sample along with the cellular protein. We used in this regard a guanidinium salt such as guanidinum thiocyanate and or ammonium sulfate associated or not with a ribonuclease inhibitor (EDTA <10 mM).

Preferred biological targets to be detected in crude saliva are Capnocytophaga gingivalis (ATCC 33624, Genbank Accession N^{o} AF543295), Prevotella melaminogenica (ATCC 25845, Genbank Accession N^{o} AJ555137), Streptococcus mitis (ATCC 15914, Genbank Accession N^{o} AJ617805,) Micrococcus luteus (ATCC 53598D, Genbank Accession N^{o} AM285006), human papillomavirus 16 (ATCC 45113, Genbank Accession N^{o} EF422141), human papillomavirus 18 (ATCC 45152, Genbank Accession N^{o} EF422111), SSAT mRNA (Genbank Accession N°NM002970), H3F3A mRNA (Genbank Accession N^{o} NM002107), IL8 mRNA (Genbank Accession N^{o} NM000584).

Beta actine mRNA (Genbank Accession N^{o} X00351) is used as internal control for calibration and quantification.

After obtaining bacteria, virus, human cells and extracellular human total nucleic acid from a saliva sample in a sterile device, the preservation buffer is added into the saliva sample at room temperature. The buffer is preferably able to open prokaryotic and eukaryotic cells, associated with preservation of total nucleic acids by action of blocking nucleases activities and precipitation of total nucleic acids. The preservation reagent is provided in dry format in a sterile polyethylene terephtalate (PET) plastic tube. Stabilizing reagent is calibrated to draw up to 3 ml of saliva associated with a dilution buffer. Calibrated and known nucleic acids (DNA or RNA), for example can be added to the collect vial in order to measure the exact quantity of saliva collected and analyzed by comparison with external standard calibration curve obtained after extraction and Reverse Transcriptase PCR quantification (some of the biotargets measured are expressed in genomic quantity per ml of saliva). Calibrated and known nucleic acids (DNA or RNA) added to the collect vial will also permit to verify the global performance of the full analytical process. Total nucleic acids are purified with a Nucleic Acid affinity resin. Our preferred system used coated paramagnetic beads compatible with the guanidinium salt having a rate of recovery of total nucleic acids from crude sample up to 90% and no selection between RNA and DNA.

The basic RT PCR process is carried out as follows. The RNA present in the total nucleic acid contained in the sample may be first reverse transcribed into cDNA (using enzyme like Tth DNA polymerase as purified enzyme and a oligonucleotide or PNA or modified oligonucleotide), and then denatured, using physical means, which are known to those of skill in the art. A preferred physical means for strand separation involves heating the nucleic acid until it is completely (>99%) denatured. Methods for the amplification of RNA targets using a thermostable DNA polymerase are described in WO9109944 incorporated herein by reference. The denatured DNA strands are then incubated in the same tube with the selected oligonucleotide primers under hybridization conditions, conditions which enable the binding of the primers to the single DNA strands. As known in the art, the primers are selected so that their relative positions along a duplex sequence are such that an extension product synthesized from one primer, when it is separated from its complement, serves as a template for the extension of the other primer to yield a replicate chain of defined length. The primer must be sufficiently long to prime the synthesis of extension products in the presence of the agent for polymerization. The exact length of the primers will depend on many factors, including temperature, source of the primer and use of the method.

Preferred oligonucleotide primers for use in the present invention are selected from the group consisting of SEQ ID No 1 to SEQ ID No 31.

Template-dependent extension of the oligonucleotide primer (s) is then catalyzed by the polymerizing agent (in the presence of adequate amounts of the four deoxyribonucleoside triphosphates (dATP, dGTP, dCTP, and dTTP or analogs), in a reaction medium which is comprised of the appropriate salts, metal cations, and pH buffering system. The products of the synthesis are duplex molecules consisting of the template strands and the primer extension strands, which include the target sequence. These products, in turn, serve as templates for another round of replication. In the second round of replication, the primer extension strand of the first cycle is annealed with its complementary primer; synthesis yields a “short” product which is bounded on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary, and is determined by the function which the product polynucleotide is to serve.

The PCR method is performed in a fashion where all of the reagents are added simultaneously, in one step. In a preferred method, the RT PCR reaction is carried out as an automated process which utilizes a thermostable enzyme like Tth. In a preferred method, the RT PCR reaction is performed in a types of thermocycler having capability for reading at least 4 different florescence dyes and developed/manufactured for real time PCR assays and commercial use.

Those skilled in the art will also be aware of the problems of contamination of a PCR by the nucleic acid from bacteria previously present in water used for buffer and resulting in non specific amplification or background. Methods to reduce these problems are provided by using adequate buffer, reagents and enzymes to avoid nucleic acid strand fragments with a size higher than 100 bp. All reagents used in the RT PCR reaction have to be processed before using. During amplification by PCR, the target polynucleotides may be detected directly by hybridization with a probe polynucleotide which forms a stable hybrid with the target sequence under high stringency to low stringency hybridization and washing conditions. Probes are typically labeled with non-radioactive labeling systems, such as fluoresceins and derivated systems.

Reverse Transcriptase activity and with DNA-dependent Polymerase activity, allowing the combination of RT and PCR in a single-tube reaction, such as Tth DNA polymerase or an enzyme like Tth DNA polymerase, and polynucleotide primers with a nucleotide sequence selected from the group consisting of SEQ ID No 2 SEQ ID No 4 SEQ ID No 5 SEQ ID No 8 SEQ ID No 10 SEQ ID No 12 SEQ ID No 14 SEQ ID No 16 SEQ ID No 18 SEQ ID No 20 SEQ ID No 21 SEQ ID No 23 SEQ ID No 25 SEQ ID No 27 SEQ ID No 29 and SEQ ID No 31 under conditions which allow hybridization of the polynucleotide to the ribonucleotide target region and Reverse Transcriptase activity of the said polymerase, or enzyme like Tth, for cDNA synthesis; and (c) amplified the cDNAs formed to a detectable level by Polymerase Chain Reaction with said polymerase enzyme like Tth DNA polymerase and polynucleotide primers and probes with a nucleotide sequence selected from the group consisting of SEQ ID No 1 to SEQ ID No 60.

More particularly, the preferred combination of primers and probes used to detect bacteria, virus and human mRNA consists of the sequences:

Prevotella melaminogenica (ATCC 25845, Genbank Accession N^{o} AJ 555137)

Seq ID No 1+Seq ID No 2+Seq ID No 32, or Seq ID No 1+Seq ID No 2+Seq ID No 49, or Seq ID No 3+Seq ID No 2+Seq ID No 49, or Seq ID No 1+Seq ID No 4+Seq ID No 32, or Seq ID No 3+Seq ID No 5+Seq ID No 49

Streptococcus mitis (ATCC 15914, Genbank Accession N^{o} AJ617805,)

Seq ID No 6+Seq ID No 8+Seq ID No 50 or Seq ID No 7+Seq ID No 8+Seq ID No 33 or Seq ID No 7+Seq ID No 8+Seq ID No 51

Capnocytophaga gingivalis (ATCC 33624, Genbank Accession AF543295)

Seq ID No 9+Seq ID No 10+Seq ID No 34 or Seq ID No 9+Seq ID No 10+Seq ID No 52

Micrococcus luteus (ATCC 53598D, Genbank Accession N^{o} AM285006)

Seq ID No 11+Seq ID No 12+Seq ID No 35, or Seq ID No 11+Seq ID No 12+Seq ID No 53

Human papillomavirus 16 (ATCC 45113, Genbank Accession N^{o} EF422141),

Seq ID No 13+Seq ID No 14+Seq ID No 36

Human papillomavirus 18 (ATCC 45152, Genbank Accession N^{o} EF422111)

Seq ID No 15+Seq ID No 16+Seq ID No 37

Beta actine mRNA (Genbank Accession N^{o} X00351)

Seq ID No 17+Seq ID No 18+Seq ID No 38 or Seq ID No 17+Seq ID No 18+Seq ID No 39 or Seq ID No 17+Seq ID No 18+Seq ID No 54 or Seq ID No 17+Seq ID No 18+Seq ID No 55 or Seq ID No 19+Seq ID No 20+Seq ID No 40 or Seq ID No 19+Seq ID No 20+Seq ID No 41 or Seq ID No 19+Seq ID No 20+Seq ID No 56

SSAT mRNA (Genbank Accession N^{o} NM002970)

Seq ID No 22+Seq ID No 23+Seq ID No 42 or Seq ID No 22+Seq ID No 23+Seq ID No 57 or Seq ID No 24+Seq ID No 25+Seq ID No 43

H3F3A mRNA (Genbank Accession N^{o} NM002107

Seq ID No 26+Seq ID No 27+Seq ID No 44 or Seq ID No 26+Seq ID No 27+Seq ID No 58 or Seq ID No 26+Seq ID No 27+Seq ID No 45 or Seq ID No 28+Seq ID No 29+Seq ID No 46 or Seq ID No 28+Seq ID No 29+Seq ID No 59

IL8 mRNA (Genbank Accession N^{o} NM000584)

Seq ID No 30+Seq ID No 31+Seq ID No 47 or Seq ID No 30+Seq ID No 31+Seq ID No 48 or Seq ID No 30+Seq ID No 31+Seq ID No 60

Also, according to the invention, the preferred probes used to detect bacteria, virus and human mRNA fixed on a microarray surface are selected from the group consisting of SEQ ID No 32 to SEQ ID No 60.

### Example 3

### Analysis of Genetic Markers in the Fluid Fraction by More than 2 Separate Steps

1—The saliva sample (up to 1000 μL) is mixed with a diluting buffer (sterile nuclease free reagent) and passed through a sterile polyethylene terephtalate (PET) plastic tube. Preservative reagent and a known nucleic acid (pure synthetic ribonucleotide) are calibrated to draw up to 3 ml of saliva associated with the dilution buffer. Full process should be realized in less than 2 minutes. This process permits immediate preservation of total nucleic acids at room temperature for up to 10 days to allow transportation delays via regular mail to laboratory.
2—Lysis at laboratory, transfer up to 1000 μl of liquid from the PET plastic into a 2 mL sterile tube with up to 1 mL of lysis buffer and then incubate at 35° C.+/−2° C. for up to one hour.
3—The lysat is processed for total nucleic acids purification with magnetic silica or polystyrene beads or funnel-design having silica membrane in mini prep spin columns able to concentrate circulating nucleic acid from plasma. The elution volume is up to 100 μl. 5-2 μL (up to 5 μL) of pure nucleic acids extract is used for the one step real time RT-PCR (RotorGene) with enzyme like Tth and the following program with Taqman Probe: I: Reverse transcription 61° C./20 min (20° C./sec) II: Denaturation 95° C./30 seconds (20° C./sec) III: PCR (35 cycles) 95° C./5 seconds (20° C./sec) 60° C./30 seconds (20° C./sec). The emitted fluorescence is measured at the end of the 60° C.

### Example 4

### Analysis of the Fluid Fraction of Saliva Sample Using Microarrays

1—The saliva sample (up to 1000 μL) is mixed with a diluting buffer (sterile nuclease free water) and passed through a sterile polyethylene terephtalate (PET) plastic tube. Preservative reagent and a known nucleic acid (pure synthetic ribonucleotide) are calibrated to draw up to 3 ml of saliva associated with the dilution buffer. Full process should be realized in less than 2 minutes. This process permits immediate preservation of total nucleic acid at room temperature for up to 10 days to allow transportation delays via regular mail to laboratory.
2—Lysis at laboratory, transfer up to 1000 μl of liquid from the PET plastic into a 2 mL sterile tube with up to 1 mL of lysis buffer and then incubate at 35° C.+/−2° C. for up to one hour.
3—The lysat is processed for total nucleic acids purification with magnetic silica or polystyrene beads or funnel-design having silica membrane in mini prep spin columns able to concentrate circulating nucleic acid from plasma. The elution volume is up to 100 μl. 5-2 μL (up to 5 μL) of pure nucleic acids extract is used for the one step RT-PCR with enzyme like Tth and the following program without Probes: I: Reverse transcription 61° C./20 min (20° C./sec) II: Denaturation 95° C./30 seconds III: PCR (35 cycles) 95° C./20 seconds, 60° C./20 seconds, 72° C./30 seconds.

Primers are Cy5 fluorescence labeled. Probes have been coated on surface of a PET slide using oligonucleotide arms.

5 μl of amplified DNA is mixed in 30 μL of hybridization buffer and incubated 2 minutes at 95° C. 25 μl of the denatured amplified DNA is hybridized on the microarray at room temperature during 10 minutes, briefly washed in two washing buffer. The microarray is dried and fluorescence is measured on a scanner and compared to standards.

### Example 5

### Analysis of the Fluid Fraction of Saliva by Using Real Time PCR Point of Care Instrument Associated to a Microfluidics Cartridge Able to Extract and Amplify Total Nucleic Acids (GeneXpert™ Solution from Cepheid Inc or Liat™ System from Iquum Inc)

1—The saliva sample (up to 1000 μL) is mixed with a diluting buffer (sterile nuclease free water) and passed through a sterile polyethylene terephtalate (PET) plastic tube. Stabilizing reagent and a know nucleic acid (pure synthetic ribonucleotide) are calibrated to draw up to 3 ml of saliva associated with the dilution buffer.

Full process should be realized in less than 2 minutes.

This process permits immediate preservation of total nucleic acid at room temperature for up to 10 days to allow transportation delays via regular mail to laboratory or direct analysis in the dental of physician office.

2—Up to 3 ml or 5 ml of saliva in stabilizing reagent is transferred in the microfluidics cartridge chamber having lysis buffer able to process for total nucleic acid purification with polystyrene beads and/or associated to physical and/or mechanical lysis. Without technician intervention, 2 μL (up to 5 μL) of pure nucleic acids extracted are transferred in the one step real time RT-PCR chamber having enzyme like Tth and associated mix PCR reagents and the following program
with Taqman Probe: I: Reverse transcription 61° C./20 min (20° C./sec) II: Denaturation 95° C./30 seconds (20° C./sec) III: PCR (35 cycles) 95° C./5 seconds (20° C./sec) 60° C./30 seconds (20° C./sec). The emitted fluorescence is measured at the end of the 60° C.

### Example 6

### Evaluation of the Overexpression of Several Genetic Marker in the Saliva of Oral Cancer Patients by Quantitative PCR

1—The saliva sample (up to 1000 μL) is mixed with a diluting buffer (sterile nuclease free water) and passed through a sterile polyethylene terephtalate (PET) plastic tube. Preservative reagent and a known nucleic acid (pure synthetic ribonucleotide) are calibrated to draw up to 3 ml of saliva associated with the dilution buffer. Full process should be realized in less than 2 minutes. This process permits immediate preservation of total nucleic acid at room temperature for up to 10 days to allow transportation delays via regular mail to laboratory.
2—Lysis at laboratory, transfer 250 μl of liquid from the PET plastic into a 2 mL sterile tube with Proteinase K and then incubate at 56° C.+/−2° C. for up to one hour. Incubate the pre-lysed sample with a lysis buffer, 10 minutes at 70° C.+/−2° C.
3—The lysat is processed for total nucleic acids purification with funnel-design having silica membrane in mini prep spin columns able to concentrate nucleic acid. The elution volume is up to 100 μL. 2 μL (up to 5 μL) of pure nucleic acids extract is used for one step RT-PCRs with enzyme like Tth and for one step PCRs with enzyme like DNA Polymerase. The following program is performed for the one step RT-PCR: I: Reverse transcription 48° C./15 min II: Denaturation 95° C./10 minutes III: PCR (40 cycles) 95° C./15 seconds, 60° C./60 seconds. The following program is performed for the one step PCR: I: Denaturation 95° C./30 seconds III: PCR (45 cycles) 95° C./10 seconds, 60° C./10 seconds, 72° C./35 seconds.

One step PCRs, using the primers SEQ ID No 1, SEQ ID No 2, SEQ ID No 7, SEQ ID No 8, SEQ ID No 9 and SEQ ID No 10 allow to amplified the DNA from Prevotella melaminogenica, Streptococcus mitis and Capnocytophaga gingivalis respectively. The detection and quantification system is a Syber Green system or a a probe system using the SEQ ID No 32 or SEQ ID No 49 for Prevotella melaminogenica quantification, SEQ ID No 33 or SEQ ID No 51 for Streptococcus mitis quantification and SEQ ID No No 34 or SEQ ID No 52 for Capnocytophaga gingivalis quantification.

One step RT-PCRs, using the primers SEQ ID No 26, SEQ ID No 27 or SEQ ID No 28, SEQ ID No 29 and SEQ ID No 22 and SEQ ID No 23 allow to amplify H3F3A and SSAT mRNA respectively. The detection and quantification system could a Syber Green system or a probe system using the SEQ ID No 44 or SEQ ID No 58 or SEQ ID No 45 or SEQ ID No 46 or SEQ ID No 59 for H3F3A quantification and SEQ ID No 42 or SEQ ID No 57 for SSAT quantification. Each assay is confirmed using commercial probe kits (ABI) that amplified the H3F3A gene NM005324 on the exon 4 and the SSAT gene NM 002970 on the exons 3 and 4.

The results obtained for the patients from the oral cancer population versus the healthy individuals are presented in table 1.

TABLE 1
overexpression of genetic biomarker in oral cancer population

| | Overexpression |
|---|---|
| Genetic biomarker | in oral cancer |
| name | population |
| Streptococcus mitis | 2.80X |
| Capnocytophaga | 2.00X |
| gingivalis | |
| Prevotella | 2.30X |
| melaninogenica | |
| SSAT | 2.63X |
| H3F3A | 2.56X |

It has been concluded from these data that the “detection” of a certain genetic marker (DNA or RNA) in a saliva sample of a patient means that said sample contains at least 2 fold the amount of said marker in the normal population.

Importantly, the tumorigenic status of 11 samples among 17 has been detected using the genetic markers SSAT, of H3F3A and the sequence of the bacteria Streptococcus mitis. Therefore, it can be conclude that the detection of these at least three genetic marker in the saliva of a human subject enables to diagnose an oral cancer with a sensibility of 64%. In other word, the detection of the mRNA of SSAT, the mRNA of H3F3A and the bacteria sequence of Streptococcus mitis indicates that the human subject has a risk superior to 60% of developing an oral cancer.

Accordingly, IL8 is not considered to be a significant marker for oral cancer in saliva.

### Example 7

### Analysis of Organic Volatile Compounds in the Volatile Fraction of Saliva

It is known for a while that volatile compounds can be extracted from fluidic samples from oral cavity giving the possibility to explore the saliva as material to be analyzed for pathogenic diseases (Volozhin et al. Stomatologiia (mosk), 2001; 80(1):9-12).

In the present case, 1 ml of saliva solution is placed in a glass vial with 10 μL of the standard solution with 1 ppm of three (3) standards (1-bromobutane, 1-bromobenzene and 1.4-dibromobenzene; final solution with 1 ppm prepared in pure water).

The samples are placed at room temperature during at least 1 hour before analyzes. The sample is heated at 40° C. during 10 minutes then the extraction of the volatile compounds is carried out at 40° C., using a CAR/PDMS fiber (SPME fiber assembly CAR/PDMS of 75 μm (Supelco, Bellefont, Pa., USA)), during 30 minutes. Then the analysis was performed using GC/MS. The GC injection port temperature is 280° C. The injection of the volatile molecules in GC/MS is carried out by thermal desorption of the fiber at 280° C. The separation of the volatile compounds was led with a non-polar capillary column. The column temperature program was: initial temperature of 40° C. for 5 min, then increase at 3° C./min to 230° C. for 2 min. The mass spectra are measured by electronic impact at 70 e.V.

The identification of the volatile molecules is obtained by:
comparison of the experimental indices of retention to those of the internal data bank
comparison of the experimental spectra to those of the bank Wiley 275K.

The results of the exhaustive analysis of all the volatile organic compounds found in the volatile fraction of human saliva are reported on table 2.

TABLE 2
Organic volatile compounds in volatile fraction of human saliva

| Molecule name | CAS number |
|---|---|
| 1,2-dichlorobenzene | 95-50-1 |
| disulfure de carbone | 137-26-8 |
| benzenecarboxylic acid | 65-85-0 |
| heptanoic acid | 111-14-8 |
| nonanoic acid | 112-05-0 |
| octanoic acid | 124-07-2 |
| pentanoic acid | 109-52-4 |
| 2-propenal, 2-methyl- | 78-85-3 |
| butanal | 123-72-8 |
| butanal, 3-methyl- | 590-86-3 |
| decanal | 112-31-2 |
| ethanal | 75-07-0 |
| heptanal | 111-71-7 |
| hexanal | 66-25-1 |
| hexanal, 2-ethyl- | 123-05-7 |
| nonanal | 124-19-6 |
| octanal | 124-13-0 |
| propanal, 2-methyl- | 78-84-2 |
| valeraldehyde, 4,4-dimethyl-2-methylene | 5375-28-0 |
| 7-oxabicyclo[4.1.0]heptane, 1-methyl- | 1713-33-3 |
| cyclopentane, methyl- | 96-37-7 |
| decane | 124-18-5 |
| hexane | 110-54-3 |
| methylcyclohexane | 108-87-2 |
| nonane | 111-84-2 |
| pentane, 2,2,4-trimethyl- | 540-84-1 |
| pentane, 2-methyl- | 107-83-5 |
| pentane, 3-methyl- | 96-14-0 |
| benzene | 71-43-2 |
| benzene, ethyl- | 100-41-4 |
| Butylated Hydroxytoluene | 128-37-0 |
| dehydro p-cymene | 1195-32-0 |
| dibenzofuran | 132-64-9 |
| m-xylene | 108-38-3 |
| o-xylene | 95-47-6 |
| p-cymene | 99-87-6 |
| styrene | 100-42-5 |
| toluene | 108-88-3 |
| 1-hexene, 3,5,5-trimethyl- | 4316-65-8 |
| 2-hexene, 2,5,5-trimethyl- | 40467-04-7 |
| butanoic acid, 2-methyl-, ethyl ester | 7452-79-1 |
| butanoic acid, 3-methyl-, ethyl ester | 108-64-5 |
| methyl thiolacetate | 1534-08-3 |
| ethyl butanoate | 105-54-4 |
| ethyl propanoate | 105-37-3 |
| ethyl-N-methylcarbamate | 105-40-8 |
| methylacetate | 79-20-9 |
| 1,3-dioxolane, 2-methyl- | 497-26-7 |
| tert-butyl ethyl ether | 637-92-3 |
| bromoethane | 74-96-4 |
| bromomethane | 74-83-9 |
| chlorobutanol | 57-15-8 |
| dibromomethane | 74-95-3 |
| dichloromethane | 75-09-2 |
| heptane, 3-bromo- | 1974-05-6 |
| hexane, 1-chloro- | 544-10-5 |
| hexane, 3-chloro- | 2346-81-8 |
| phenol, 2-chloro-4-(1,1-dimethylpropyl)- | 98-28-2 |
| tribromomethane | 75-25-2 |
| 1H-pyrrole | 109-97-7 |
| 1H-pyrrole, 1-methyl- | 96-54-8 |
| 2-furfural | 98-01-1 |
| furan, 2-acetyl- | 1192-62-7 |
| furan, 2-ethyl- | 3208-16-0 |
| furan, 2-pentyl- | 3777-69-3 |
| furan, 3-methyl | 930-27-8 |
| furan, 5-methyl-2-propionyl- | 33978-70-0 |
| pyrazine | 290-37-9 |
| pyrazine, 2,5-dimethyl- | 123-32-0 |
| pyrazine, ethyl- | 13925-00-3 |
| pyrazine, methyl- | 109-08-0 |
| pyridine | 110-86-1 |
| 2-butenenitrile | 4786-20-3 |
| 2-piperidinone | 675-20-7 |
| 3-butenenitrile | 109-75-1 |
| benzene isocyanato | 103-71-9 |
| benzonitrile | 100-47-0 |
| butanenitrile, 3-methyl- | 625-28-5 |
| ethane, isocyano- | 624-79-3 |
| ethyl isocyanate | 109-90-0 |
| propanenitrile | 107-12-0 |
| propanenitrile, 2-methyl- | 78-82-0 |
| 1-hexanol | 111-27-3 |
| 1-hexanol, 2-ethyl- | 104-76-7 |
| 1-pentanol | 71-41-0 |
| 1-propanol, 2-methyl- | 78-83-1 |
| 2-butanol | 15892-23-6 |
| 2-butanol, 2-methyl- | 75-85-4 |
| 2-hexanol, 2,5-dimethyl- | 3730-60-7 |
| 2-pentanol, 2-methyl- | 590-36-3 |
| 2-propanol, 2-methyl- | 75-65-0 |
| 3-hexanol | 623-37-0 |
| ethanol | 64-17-5 |
| phenol | 108-95-2 |
| phenol, 4-(1,1-dimethylpropyl)- | 80-46-6 |
| skatole | 83-34-1 |
| 1,3-isobenzofuranedione | 85-44-9 |
| 2,3-octanedione | 585-25-1 |
| 2,4-pentanedione, 3-methyl- | 815-57-6 |
| 2,6-di-tert-butyl-p-benzoquinone | 719-22-2 |
| 2-butanone | 78-93-3 |
| 2-butanone, 3,3-dimethyl- | 75-97-8 |
| 2-butanone, 3-hydroxy- | 513-86-0 |
| 2-cyclohexen-1-one, 3,4,4-trimethyl- | 17299-41-1 |
| 2-cyclopenten-1-one, 2,3-dimethyl- | 1121-05-7 |
| 2-cyclopenten-1-one, 3-methyl- | 2758-18-1 |
| 2-hexanone, 3-methyl- | 2550-21-2 |
| 2-methyl-2-cyclopenten-1-one | 1120-73-6 |
| 3-heptanone | 106-35-4 |
| 3-hepten-2-one, 5-methyl- | 5090-16-4 |
| 3-octanone | 106-68-3 |
| 3-penten-2-one, 3-methyl- | 565-62-8 |
| 5-hepten-2-one, 6-methyl- | 110-93-0 |
| acetophenone | 98-86-2 |
| cyclohexanone | 108-94-1 |
| cyclopentanone, 2-methyl- | 1120-72-5 |
| cyclopentanone, 3-methyl- | 1757-42-2 |
| methylbutanone | 563-80-4 |
| p-methylacetophenone | 122-00-9 |
| disulfide, dimethyl | 624-92-0 |
| methanethiol | 74-93-1 |
| sulfone, dimethyl- | 67-71-0 |
| dimethyl sulfide | 75-18-3 |
| (Z)-caryophyllene | 118-45-0 |
| anethole (E) | 4180-23-8 |
| a-pinene | 80-56-8 |
| a-terpineol | 10482-56-1 |
| a-thujene | 2867-05-2 |
| b-bourbonene | 5208-59-3 |
| b-caryophyllene | 87-44-5 |
| b-pinene | 127-91-3 |
| camphene | 79-92-5 |
| carvone | 2244-16-8 |
| cis-p-menthan-3-one | 491-07-6 |
| dihydromyrcenol | 18479-59-9 |
| eucalyptol | 470-82-6 |
| limonene | 138-86-3 |
| m-mentha-6,8-diene | 1461-27-4 |
| neo-menthol | 491-01-0 |
| piperitone | 89-81-6 |
| p-menth-3-ene | 500-00-5 |
| trans-p-menthan-3-one | 89-80-5 |
| benzaldehyde | 100-52-7 |
| 2-octanone | 111-13-7 |
| 2-heptanone | 110-43-0 |
| 2,3-pentanedione | 600-14-6 |
| 2-pentanone, 3-methyl- | 565-61-7 |
| acetic acid | 64-19-7 |
| 3-hexanone | 589-38-8 |
| 1-propene, 2-methyl- | 115-11-7 |
| benzaldehyde, 2-methyl- | 529-20-4 |
| propanoic acid | 79-09-4 |
| butanoic acid | 107-92-7 |
| 3-pentanone, 2-methyl- | 565-69-5 |
| 2-pentanone 4,4-dimethyl- | 590-50-1 |
| ethyl acetate | 141-78-6 |
| thiophene, 3-methyl- | 616-44-4 |
| 2-pentanone | 107-87-9 |
| 2-hexanone | 591-78-6 |
| isovaleric acid | 503-74-2 |
| 2-pentanone, 4-methyl- | 108-10-1 |
| 2-butene, 2-methyl- | 513-35-9 |
| 1-propanol | 71-23-8 |
| 2-butenal, 2-methyl (E) | 497-03-0 |
| butanoic acid, 2-methyl- | 116-53-0 |
| 2-propanol | 67-63-0 |
| isobutyric acid | 79-31-2 |
| benzene, 2-methyl-1-propenyl- | 768-49-0 |
| hexanoic acid | 142-62-1 |
| acetone | 67-64-1 |
| pentanoic acid, 4-methyl- | 646-07-1 |
| cyclopropane, 1,2-dimethyl (cis) | 930-18-7 |
| p-cresol | 106-44-5 |
| 2,3-butanedione | 431-03-8 |
| 1-butanol, 3-methyl- | 123-51-3 |
| m-methyl acetophenone | 585-74-0 |
| 3-hexen-2-one, 5-methyl- | 5166-53-0 |
| indole | 120-72-9 |
| 3-hexen-2-one, 3,4-dimethyl- | 1635-02-5 |
| pentanal | 110-62-3 |
| 2-pentanol, 2,3-dimethyl- | 4911-70-0 |
| m-cymene | 535-77-3 |
| cyclohexanone | 108-94-1 |
| aniline | 62-53-3 |
| furan, 2-methyl- | 534-22-5 |
| 3-pentanone, 2,4 dimethyl- | 565-80-0 |
| 3-buten-2-one 3-methyl | 814-78-8 |
| hexanenitrile | 628-73-9 |
| heptanenitrile | 629-08-3 |
| pentane nitrile | 110-59-8 |
| butanenitrile | 109-74-0 |
| acrylonitrile | 107-13-1 |

From our experimental studies, 192 volatile molecules have been identified in the volatile fraction of human saliva (table 2). Principal volatile compounds identified in saliva are ketones, acids, aldehydes, alcohols and aromatic compounds.

Among these compounds, 57 volatile compounds have been preselected to be used has possible biomarkers discriminating factor for oral cancer early detection (table 3).

TABLE 3
Volatile compounds potentially indicative of oral cancer susceptibility

| Molecule name | CAS Number |
|---|---|
| benzaldehyde | 100-52-7 |
| 2-octanone | 111-13-7 |
| 2-heptanone | 110-43-0 |
| 2,3-pentanedione | 600-14-6 |
| 3-methyl-2-pentanone | 565-61-7 |
| acetic acid | 64-19-7 |
| 3-hexanone | 589-38-8 |
| 2-methyl-1-propene | 115-11-7 |
| 2-methyl-benzaldehyde | 529-20-4 |
| propanoic acid | 79-09-4 |
| butanoic acid | 107-92-7 |
| 2-methyl-3-pentanone | 565-69-5 |
| 4,4-dimethyl-2-pentanone | 590-50-1 |
| ethyl acetate | 141-78-6 |
| 3-methyl-thiophene | 616-44-4 |
| 2-pentanone | 107-87-9 |
| 2-hexanone | 591-78-6 |
| isovaleric acid | 503-74-2 |
| 4-methyl-2-pentanone | 108-10-1 |
| 2-methyl-2-butene | 513-35-9 |
| 1-propanol | 71-23-8 |
| (E) 2-methyl-2-butenal | 497-03-0 |
| 2-methyl-butanoic acid | 116-53-0 |
| 2-propanol | 67-63-0 |
| isobutyric acid | 79-31-2 |
| 2-methyl-1-propenyl-benzene | 768-49-0 |
| hexanoic acid | 142-62-1 |
| acetone | 67-64-1 |
| 4-methyl-pentanoic acid | 646-07-1 |
| (cis) 1,2-dimethyl cyclopropane | 930-18-7 |
| p-cresol | 106-44-5 |
| 2,3-butanedione | 431-03-8 |
| 3-methyl-1-butanol | 123-51-3 |
| m-methyl acetophenone | 585-74-0 |
| 5-methyl-3-hexen-2-one | 5166-53-0 |
| indole | 120-72-9 |
| 3,4-dimethyl-3-hexen-2-one | 1635-02-5 |
| pentanal | 110-62-3 |
| 2,3-dimethyl-2-pentanol | 4911-70-0 |
| m-cymene | 535-77-3 |
| cyclohexanone | 108-94-1 |
| aniline | 62-53-3 |
| 2-methyl-furan | 534-22-5 |
| 2,4 dimethyl-3-pentanone | 565-80-0 |
| 3-methyl-3-buten-2-one | 814-78-8 |
| hexanenitrile | 628-73-9 |
| heptanenitrile | 629-08-3 |
| pentane nitrile | 110-59-8 |
| butanenitrile | 109-74-0 |
| 3-methyl-2-pentanone | 565-61-7 |
| methylbutanone | 563-80-4 |
| butanal | 123-72-8 |
| hexanal | 66-25-1 |
| phenol | 108-95-2 |
| m-xylene | 108-38-3 |
| p-xylene | 106-42-3 |
| ethanal | 75-07-0 |
| benzene | 71-43-2 |
| acrylonitrile | 107-13-1 |

According to our results, at least 19 of these 57 compounds are indeed correlated with oral cancer, as shown below.

### Example 8

### Quantification of the Identified Volatile Compounds in Cancer/Healthy Patients

The quantification of the volatile compounds is made by comparison with standard controls that have been added in the preservation buffer at the beginning of the experiment. In this case, the followings molecular standards have been used:
1-bromobutane (CAS number 109-65-9)
1-bromobenzene (CAS number 108-86-1)
1,4-dibromobenzene (CAS number 106-37-6)

TABLE 4
Particular biomarkers indicative of oral cancer predisposition

| | Overexpression in | Overexpression in |
|---|---|---|
| Biomarker name | oral cancer population | normal population |
| 2,3-pentanedione | 6.00X | |
| 3-methyltiophene | 1.50X | |
| acetone | 2.30X | |
| hexanitrile | 3.00X | |
| benzaldehyde | 1.80X | |
| 3-methyl-2- | 2.10X | |
| pentanone | | |
| 2,3-butanedione | 4.40X | |
| 2-propanol | 2.80X | |
| ethyl acetate | 3.90X | |
| 1-propanol | 1.90X | |
| hexanal | 1.60X | |
| 5-methyl-3-hexen- | 1.70X | |
| 2-one | | |
| m- and p-xylene | 1.50X | |
| 2-methyl-2-butenal (E) | 1.90X | |
| 2-methyl-2-butene | | 2.00X |
| 2-methyl-1- | | 3.30X |
| propene | | |
| (cis) 1,2-dimethyl | | 1.70X |
| cyclopropane | | |

It has been concluded from these data that the “detection” of a certain volatile compound in a saliva sample of a patient means that said sample contains at least 1.5 fold the amount of said compound in the normal population.

### Example 9

### Statistical Analysis of the Presence of Biochemical Organic Compounds in the Saliva of Oral Cancer Patient Vs Healthy Individuals

Software STATISTICA version 8.0 of StatSoft France (2007) is used for data analysis. The significances of the differences between the groups were tested by from Factorial Discriminating Analysis (FDA). Thus the similarities or the differences of the samples can be visualized graphically.

The identification of the volatile molecules is obtained by:
Comparison of the experimental indices of retention to those of the internal data bank,
comparison of the experimental spectra to those of the bank Wiley 275K and NIST 2.0d, built april 2005.

Statistical Model 1

Total population tested is 45 human subjects from two distinctive environmental geographic areas. Oral cancer population is confirmed by visual diagnostics performed by an anticancer center.

The statistical analyzes were carried out on 109 volatile compounds. Abundances of the molecules in each sample were reported to abundances of the 3 internal standards analyzed with saliva. The principal volatile compounds identified in saliva are ketones, acids, aldehydes, alcohols and aromatic compounds. All the samples have a strong abundance in hydrazoic acid coming directly from the buffer solution of conservation.

On the 108 volatile compounds, 49 are significant to separate the group “tumor” from the reference group. A discriminating factorial analysis on these variables makes it possible to classify well 97.78% of the samples with 4 volatile compounds: the hexanenitrile, the 2,3-pentanedione, 3-methylthiophene and acetone. Only 1 false-positive have been detected with the statistical model 1.

Statistical Model 2

Total population tested is 45 human subjects from two distinctive environmental geographic areas. Oral cancer population is confirmed by visual diagnostics performed by a specialized anticancer center.

The statistical analyzes were carried out on 108 volatile compounds. Abundances of the molecules in each sample were reported to abundances of the 3 internal standards analyzed with saliva. The principal volatile compounds identified in saliva are ketones, acids, aldehydes, alcohols and aromatic compounds. All the samples have a strong abundance in hydrazoic acid coming directly from the buffer solution of conservation.

All nitriles volatile compounds have been removed from the statistical model number 2. For this study, the 10 made up “nitriles” were not taken into account.

From the 98 remaining volatile compounds, an ANOVA test according to the factor “tumor” showed that 45 components are significant to separate the group “tumor” from the reference group.

A discriminating factorial analysis on these variables makes it possible to classify well 93.33% of the samples with 4 volatile compounds: benzaldehyde, acetone, the 2,3-pentanedione and 2-methyl-2-butene. The first 3 molecules are side of the group “tumor” and the 2-methyl-2-butene on the side of the control group. 3 false-negatives and no false positive have been detected with the statistical model 2.

To conclude, this study highlights the tight link existing between 14 organic compounds (namely hexanenitrile, the 2,3-pentanedione, 3-methylthiophene, 2-methyl-2-butylene, 3-methyl-2-pentanone, 2,3-butane dione, 2-propanol, ethyl acetate, 1-propanol, hexanal, 5-methyl-3-hexen-2-one, m-xylene, p-xylene, 2-methyl-2-butenal (E)) and oral cancer in human. It is noteworthy that none of them have ever been found in exhaled breath (Mashir A, Advanced Powder Technology, 2009) or being associated to oral cancer.

### Example 10

### Diagnostic Test Based on the Ratios of Specific Organic Molecules

Software STATISTICA version 8.0 of StatSoft France (2007) is used for data analysis. The significances of the differences between the groups were tested by Factorial Discriminating Analysis (FDA). Thus the similarities or the differences of the samples can be visualized graphically.

### Tested Population

Total population tested is 52 human subjects from two distinctive environmental geographic areas. Oral cancer population is confirmed by visual diagnostics performed by a specialized anticancer center.

The following volatile organic compounds are used in the diagnostic test:

3-methyl-2-pentanone (CAS number: 565-61-7)
Methylbutanone (CAS number: 563-80-4)
2.4-dimethyl-3-pentanone (CAS number: 565-80-0)
Benzene (CAS number: 71-43-2)
Phenol (CAS number: 108-95-2)
2.3-butanedione (CAS number: 431-03-8)
5-methyl-3-hexen-2-one (CAS number: 5166-53-0)
2-methyl-1-propene (CAS number: 115-11-7)
Butanal (CAS number: 123-72-8)
Hexanal (CAS number: 66-25-1)
2-propanol (CAS number: 67-63-0)
Ethyl acetate (CAS number: 141-78-6)
Hexanenitrile (CAS number: 628-73-9)
1-propanol (CAS number: 71-23-8)
(cis) 1,2-dimethyl-cyclopropane (CAS number: 930-18-7)
m- and p-xylene (CAS number: 108-38-3 and CAS number: 106-42-3)
(E) 2-methyl-2-butenal (CAS number: 497-03-0)
3-methyl-thiophene (CAS number: 616-44-4)
Ethanal (CAS number: 75-07-0)

The median values by group were calculated for the following ratios:
3-methyl-2-pentanone/methyl butanone
2.4-dimethyl-3-pentanone/benzene
phenol/2,3-butanedione
5-methyl-3-hexen-2-one/2-methyl-1-propene
butanal/hexanal
2-propanol/ethyl acetate
hexanenitrile/1-propanol
2-propanol/(cis) 1.2-dimethyl cyclopropane
m-xylene/2-methyl-2-butenal
3-methyl-2-pentanone/3-methyl-thiophene,
2,3-butanedione/ethanal

The statistical method used is FDA (Factorial Discrimination Analysis).

The median values by group were calculated for each of the ratios.

The 5 followings were found to be statistically significative of oral cancer patient group or control group:
1) 3-methyl-2-pentanone/methyl butanone (R1)
2) Butanal/hexanal (R56)
3) Hexanenitrile/1-propanol (R260)
4) 2-propanol/(cis) 1,2 dimethyl cyclopropane (R266)
5) Phenol/2,3 butanedione (R269).

Among these ratios, 2 were found to be reproducibly correlated with healthy subjects, and three were indicative of oral cancer suffering patients (table 5).

TABLE 5
Median values for the 5 ratios correlated with oral cancer or
healthiness

| | | | | R266 | |
|---|---|---|---|---|---|
| | R1 | | | 2-propanol/ | |
| | 3-methyl-2- | R56 | R260 | (cis) 1,2- | R269 |
| | pentanone/ | Butanal/ | Hexanenitrile/ | dimethyl | Phenol/2,3- |
| | methyl butanone | hexanal | 1-propanol | cyclopropane | butanedione |
| Average | 0.182 | 0.223 | 0.040 | 2.939 | 0.696 |
| healthy | | | | | |
| Average | 0.357 | 0.138 | 0.089 | 9.359 | 0.217 |
| Oral Cancer | | | | | |

The 3 ratios R1 (3-methyl-2-pentanone/methyl butanone), 8260 (Hexanenitrile/1-propanol) and R266 (Phenol/2,3-butanedione) are of the side of the oral cancer group group and the values are respectively 1.96; 2.24 and 3.18 times higher in this group than in the healthy group.

The 2 ratios R56 (Butanal/hexanal) and 8269 (Phenol/2,3-butanedione) are of the side of the healthy group and are respectively 1.62 and 3.21 higher in this group than in the oral cancer group.

The absolute limiting values of the ratios permitting to classify the patients in a potential “oral cancer group” are given in table 6:

TABLE 6
Absolute limiting ratio values permitting to classify the patients

| | Condition so that the | Nb samples |
|---|---|---|
| | sample is “oral cancer | corresponding to |
| Report/ratio | risk” | the ratio |
| 3-methyl-2-pentanone/ | >0.344 | 17 |
| methyl butanone | | |
| Butane/hexanal | <0.11 | 10 |
| Hexanenitrile/1-propanol | >0.167 | 3 |
| 2-propanol/(cis) 1,2-dimethyl | >10.33 | 9 |
| cyclopropane | | |
| Phenol/2,3-butanedione | <0.005 | 4 |

To classify the samples it is necessary to apply the following formula (from the FDA statistical method) taking into account all these 5 ratios (linear combination of the 5 variable ratios):

Factor X=1.8277−7.3472*R1−0.125*R266−14.2293*R260+1.2050*R269+8.883*R56

If factor X≦0.6, the sample is classified in the Oral Cancer Risk Population
If factor X>0.6, the sample is classified healthy

Therefore, the method of the invention, based on:
i) the recovery of the volatile fraction of the saliva of a human subject,
i) the quantification of ten biochemical organic compounds (3-methyl-2-pentanone, methyl butanone, Butanal, hexanal, Hexanenitrile, 1-propanol 2-propanol, (cis) 1,2-dimethyl cyclopropane, phenol, and 2,3-butanedione) in said volatile fraction,
ii) calculation of the ratios R1, R266, 8260, 8269 and R56 as mentioned above,
iii) calculation of said factor X and its comparison with the threshold 0.6,
   enables the man skilled in the art to prognose and/or diagnose an oral cancer in said human subject.

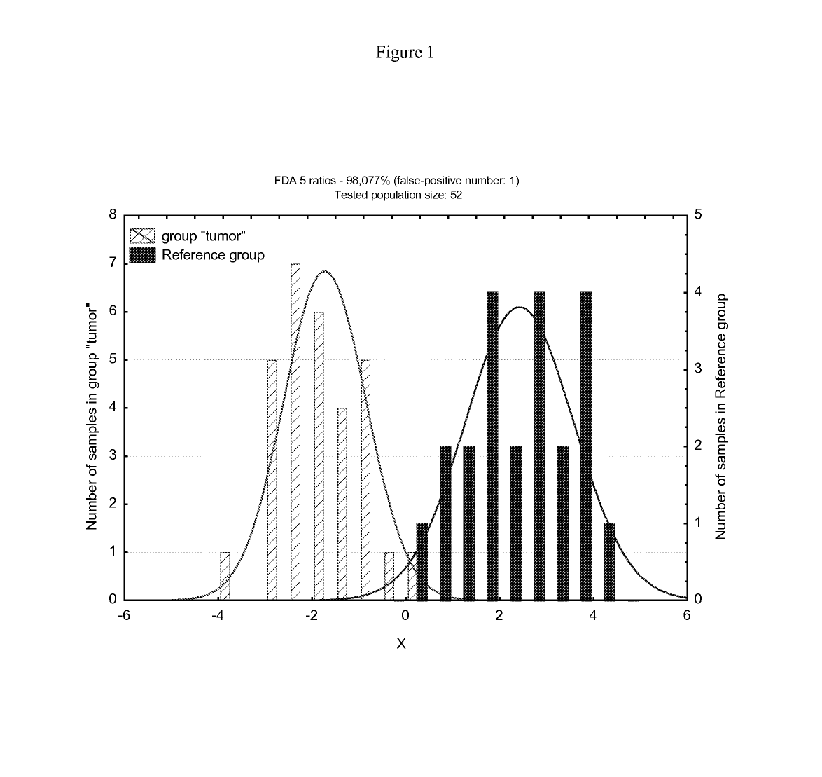

The analysis of the ratios between the organic compounds: 3-methyl-2-pentanone/methyl butanone, Butanal/hexanal, Hexanenitrile/1-propanol 2-propanol/(cis) 1,2-dimethyl cyclopropane, and Phenol/2,3-butanedione in the volatile fraction of the saliva of a human subject permits to obtain a highly sensitive test of predisposition of oral cancer (98.077% sensitivity; 1 false-positive) (FIG. 1).

### Example 11

### Combination of the Biomarkers of Fluid Fraction and Volatile Fraction for Diagnosing/Prognosing Oral Cancer

Software STATISTICA version 8.0 of StatSoft France (2007) is used for data analysis. The significances of the differences between the groups were obtained from Factorial Discriminating Analysis (FDA). Thus the similarities or the differences of the samples can be visualized graphically.

The best combination of biomarker was the following: Streptococcus mitis+SSAT+H3F3A+hexanenitrile+2.3-pentanedione, 3-methylthiophene+acetone. Indeed the use of such markers permits to obtain 100% sensibility results as shown in table 7.

TABLE 7
Sensibility of the diagnostic test based on biomarkers from the fluid
fraction and volatile fraction

| | | | volatile | | bacteria + |
|---|---|---|---|---|---|
| | | | compounds | bacteria + | hmRNA + |
| | | human | statistical | human | volatile |
| | bacteria | mRNA | model 1 | mRNA | compounds |
| Specificity % | 76 | 54 | 98 | 83 | 98 |
| Sensibility % | 79 | 32 | 100 | 89 | 100 |

1. An in vitro method of diagnosing a predisposition to oral cancer or of diagnosing an oral cancer in a human subject, the method comprising:
i) collecting and stabilizing a crude saliva sample from said human subject;
ii) analyzing a fluid fraction of said stabilized saliva sample to detect nucleic acid sequences of human papillomavirus (HPV); and
iii) diagnosing said oral cancer or said predisposition to develop oral cancer when said HPV nucleic acid sequences are detected.

2. The method of claim 1, wherein said HPV is HPV 16 or HPV 18.

3. The method of claim 1, further comprising analyzing the fluid fraction of said stabilized saliva sample to detect at least one biochemical organic compound.

4. The method of claim 1, further comprising analyzing the fluid fraction of said stabilized saliva sample to detect nucleic acid sequences of human or of bacterial origin, said nucleic acid sequences being selected from:
human sequences SSAT mRNA (SEQ ID NO: 62), H3F3A mRNA (SEQ ID NO: 63), and IL8 mRNA (SEQ ID NO: 64); and
bacterial sequences from Capnocytophaga gingivalis, Prevotella melaminogenica, Streptococcus mitis, or Micrococcus luteus.

5. The method of claim 4, wherein the detection of at least two of said human mRNA sequences, and at least one of said bacterial sequences indicates that the human subject is at high risk of developing an oral cancer.

6. The method of claim 1, wherein the crude saliva is stabilized with a solution comprising guanidium thiocyanate, ammonium sulfate, sodium azide, exonuclease inhibitor, endonuclease inhibitor, or combinations thereof.

7. The method of claim 1, wherein analyzing the fluid fraction of the stabilized saliva sample to detect the HPV nucleic acid sequences comprises incubating the sample with a thermostable enzyme having RNA-dependent reverse transcriptase activity and DNA-dependent polymerase activity.

8. The method of claim 7, wherein the incubation comprises a combination of RT-PCR and PCR performed in a single-tube reaction.

9. The method of claim 1, wherein the HPV nucleic acid sequences are detected when the expression level of said sequences is 2 fold or higher than the mean expression level of said sequences in a normal population.

10. The method of claim 1, comprising detecting at least one nucleic acid sequence selected from the group consisting of SEQ ID NO: 62-70.

11. The method of claim 1, comprising:
a) collecting a sample of crude saliva from the human subject in a sterile device;
b) stabilizing the sample by adding a solution comprising guanidinum thiocyanate, ammonium sulfate, sodium azide, exonuclease inhibitor, endonuclease inhibitor, or combinations thereof;
c) extracting total nucleic acid from the stabilized saliva sample, and precipitating and purifying the total nucleic acids;
d) incubating the purified nucleic acid with a thermostable enzyme having RNA-dependant reverse transcriptase activity and DNA-dependant polymerase activity, and with polynucleotide primers, under conditions that allow the reverse transcriptase activity of the thermostable enzyme to synthesize cDNA, and amplify genomic DNA and cDNA to a detectable level by polymerase chain reaction; and
e) detecting said HPV sequences by hybridizing the amplified nucleic acid with one or more polynucleotide probes specific to HPV 16 or HPV 18.

12. The method according to claim 11, wherein said primers and probes are selected from the group consisting of:
HPV 16 sequences of SEQ ID NO: 13, SEQ ID NO: 14, and SEQ ID NO: 36; and
HPV 18 sequences of SEQ ID NO: 15, SEQ ID NO: 16, and SEQ ID NO: 37.

13. The method of claim 11, further comprising
f) detecting nucleic acid sequences by hybridizing the amplified nucleic acid with one or more polynucleotide probes specific to:
human sequences SSAT mRNA (SEQ ID No 62), H3F3A mRNA (SEQ ID No 63) and IL8 mRNA (SEQ ID No 64); or
bacterial sequences from Capnocytophaga gingivalis, Prevotella melaminogenica, Streptococcus mitis and Micrococcus luteus.

14. A kit for conducting the method of claim 11, comprising said primers and probes and at least one of the following:
a) a sterile saliva sample collection device;
b) a spray dry preservative reagent;
c) a resin having affinity for total nucleic acid;
d) a thermostable enzyme having RNA-dependant reverse transcriptase activity and DNA-dependant polymerase activity and polynucleotide;
e) a positive control nucleic acid;
f) a collection reagent.

15. The method of claim 2, wherein said HPV 16 is ATCC ATCC 45113 and said HPV 18 is ATCC 45152.

16. The method of claim 11, wherein said HPV 16 is ATCC 45113 and said HPV 18 is ATCC 45152.

17. The method of claim 4, wherein said bacteria are Capnocytophaga gingivalis (ATCC 33624), Prevotella melaminogenica (ATCC 25845), Streptococcus mitis (ATCC 15914) and Micrococcus luteus (ATCC 53598D).

18. The method of claim 13, wherein said bacteria are Capnocytophaga gingivalis (ATCC 33624), Prevotella melaminogenica (ATCC 25845), Streptococcus mitis (ATCC 15914) and Micrococcus luteus (ATCC 53598D).

19. The method of claim 1, wherein said oral cancer or said predisposition to develop oral cancer is diagnosed when said HPV nucleic acid sequences are detected in the sample at a level that is 2 fold or higher than that of a normal population.


SEQUENCE LISTING (70 sequences)

SEQ ID NO: 1 - DNA, 20 bp, artificial sequence
  Primer forward
gtgggataacctgccgaaag

SEQ ID NO: 2 - DNA, 25 bp, artificial sequence
  Primer reverse
cccatccattaccgataaatcttta

SEQ ID NO: 3 - DNA, 21 bp, artificial sequence
  Primer forward
ggcagactaatacctgcatag

SEQ ID NO: 4 - DNA, 24 bp, artificial sequence
  Primer reverse
atccattaccgataaactttcttc

SEQ ID NO: 5 - DNA, 25 bp, artificial sequence
  Primer reverse
catccccatccattaccgataaatc

SEQ ID NO: 6 - DNA, 21 bp, artificial sequence
  Primer forward
taccgcataagagtagatgtt

SEQ ID NO: 7 - DNA, 21 bp, artificial sequence
  Primer forward
cgatagctaataccgcataag

SEQ ID NO: 8 - DNA, 21 bp, artificial sequence
  Primer reverse
caggtccatctggtagtgatg

SEQ ID NO: 9 - DNA, 23 bp, artificial sequence
  Primer forward
ggatagcccggagaaatttggat

SEQ ID NO: 10 - DNA, 27 bp, artificial sequence
  Primer reverse
cgtcatcaaagtacacgtactccttat

SEQ ID NO: 11 - DNA, 21 bp, artificial sequence
  Primer forward
taccggataggagcgtccacc

SEQ ID NO: 12 - DNA, 21 bp, artificial sequence
  Primer reverse
taggccgcgagtccatccaaa

SEQ ID NO: 13 - DNA, 25 bp, artificial sequence
  Primer forward
ttgcagatcatcaagaacacgtaga

SEQ ID NO: 14 - DNA, 27 bp, artificial sequence
  Primer reverse
cagtagagatcagttgtctctggttgc

SEQ ID NO: 15 - DNA, 19 bp, artificial sequence
  Primer forward
agaggccagtgccattcgt

SEQ ID NO: 16 - DNA, 21 bp, artificial sequence
  Primer reverse
gtttctctgcgtcgttggagt

SEQ ID NO: 17 - DNA, 18 bp, artificial sequence
  Primer forward
cgtcttcccctccatcgt

SEQ ID NO: 18 - DNA, 23 bp, artificial sequence
  Primer reverse
agctcattgtagaaggtgtggtg

SEQ ID NO: 19 - DNA, 21 bp, artificial sequence
  Primer forward
aagacctgtacgccaacacag

SEQ ID NO: 20 - DNA, 20 bp, artificial sequence
  Primer reverse
cgtcatactcctgcttgctg

SEQ ID NO: 21 - DNA, 22 bp, artificial sequence
  Primer reverse
atactcctgcttgctgatccac

SEQ ID NO: 22 - DNA, 21 bp, artificial sequence
  Primer forward
ggatcagaaattctgaagaat

SEQ ID NO: 23 - DNA, 21 bp, artificial sequence
  Primer reverse
accctcttcactggacagatc

SEQ ID NO: 24 - DNA, 21 bp, artificial sequence
  Primer forward
ccagtgaagagggttggagac

SEQ ID NO: 25 - DNA, 22 bp, artificial sequence
  Primer reverse
tggaggttgtcatctacagcag

SEQ ID NO: 26 - DNA, 20 bp, artificial sequence
  Primer forward
ggcgctccgtgaaattagac

SEQ ID NO: 27 - DNA, 19 bp, artificial sequence
  Primer reverse
cgctggaagggaagtttgc

SEQ ID NO: 28 - DNA, 19 bp, artificial sequence
  Primer forward
aaagcacccaggaagcaac

SEQ ID NO: 29 - DNA, 22 bp, artificial sequence
  Primer reverse
gcgaatcagaagttcagtggac

SEQ ID NO: 30 - DNA, 22 bp, artificial sequence
  Primer forward
gagggttgtggagaagtttttg

SEQ ID NO: 31 - DNA, 22 bp, artificial sequence
  Primer reverse
ctggcatcttcactgattcttg

SEQ ID NO: 32 - DNA, 30 bp, artificial sequence
  Probe forward
gcatagtcttcgatgacggcatcagatttg

SEQ ID NO: 33 - DNA, 21 bp, artificial sequence
  Probe forward
gatgttccatgacatttrctt

SEQ ID NO: 34 - DNA, 21 bp, artificial sequence
  Probe forward
attggatggcatcatttgata

SEQ ID NO: 35 - DNA, 18 bp, artificial sequence
  Probe forward
catggtgggtgttggaaa

SEQ ID NO: 36 - DNA, 33 bp, artificial sequence
  Probe forward
aatcatgcatggagatacacctacattgcatga

SEQ ID NO: 37 - DNA, 22 bp, artificial sequence
  Probe forward
gctgcaaccgagcacgacagga

SEQ ID NO: 38 - DNA, 18 bp, artificial sequence
  Probe forward
caggcaccagggcgtgat

SEQ ID NO: 39 - DNA, 16 bp, artificial sequence
  Probe forward
cgagcacggcatcgtc

SEQ ID NO: 40 - DNA, 18 bp, artificial sequence
  Probe forward
gcagatgtggatcagcaa

SEQ ID NO: 41 - DNA, 22 bp, artificial sequence
  Probe forward
aggatgcagaaggagatcactg

SEQ ID NO: 42 - DNA, 21 bp, artificial sequence
  Probe forward
taagccaggttgcaatgaggt

SEQ ID NO: 43 - DNA, 21 bp, artificial sequence
  Probe forward
gcactcctcactcctctgttg

SEQ ID NO: 44 - DNA, 26 bp, artificial sequence
  Probe forward
tatcagaagtccactgaacttctgat

SEQ ID NO: 45 - DNA, 29 bp, artificial sequence
  Probe forward
atcagaagtccactgaacttctgatycgc

SEQ ID NO: 46 - DNA, 20 bp, artificial sequence
  Probe forward
ggcgctccgtgaaattagac

SEQ ID NO: 47 - DNA, 19 bp, artificial sequence
  Probe forward
ttcattctctgtggtatcc

SEQ ID NO: 48 - DNA, 17 bp, artificial sequence
  Probe forward
ttcattctctgtggtat

SEQ ID NO: 49 - DNA, 30 bp, artificial sequence
  Probe reverse
caaatctgatgccgtcatcgaagactatgc

SEQ ID NO: 50 - DNA, 21 bp, artificial sequence
  Probe reverse
aagyaaatgtcatggaacatc

SEQ ID NO: 51 - DNA, 21 bp, artificial sequence
  Probe reverse
aagyaaatgtcatggaacatc

SEQ ID NO: 52 - DNA, 21 bp, artificial sequence
  Probe reverse
tatcaaatgatgccatccaat

SEQ ID NO: 53 - DNA, 18 bp, artificial sequence
  Probe reverse
tttccaacacccaccatg

SEQ ID NO: 54 - DNA, 18 bp, artificial sequence
  Probe reverse
atcacgccctggtgcctg

SEQ ID NO: 55 - DNA, 16 bp, artificial sequence
  Probe reverse
gacgatgccgtgctcg

SEQ ID NO: 56 - DNA, 22 bp, artificial sequence
  Probe reverse
cagtgatctccttctgcatcct

SEQ ID NO: 57 - DNA, 21 bp, artificial sequence
  Probe reverse
acctcattgcaacctggctta

SEQ ID NO: 58 - DNA, 26 bp, artificial sequence
  Probe reverse
atcagaagttcagtggacttctgata

SEQ ID NO: 59 - DNA, 20 bp, artificial sequence
  Probe reverse
gtctaatttcacggagcgcc

SEQ ID NO: 60 - DNA, 17 bp, artificial sequence
  Probe reverse
ataccacagagaatgaa

SEQ ID NO: 61 - DNA, 1761 bp, Homo sapiens
  misc_feature: Human mRNA for beta-actin - NCBI X00351
ttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 62 - DNA, 1060 bp, Homo sapiens
  misc_feature: Homo sapiens spermidine/spermine N1- acetyltransferase 1 (SAT1), mRNA NCBI NM_002970
cgcgggccgactggtgtttatccgtcactcgccgaggttccttgggtcatggtgccagcctgactgagaagaggacgctcccgggagacgaatgaggaaccacctcctcctactgttcaagtacaggggcctggtccgcaaagggaagaaaagcaaaagacgaaaatggctaaattcgtgatccgcccagccactgccgccgactgcagtgacatactgcggctgatcaaggagctggctaaatatgaatacatggaagaacaagtaatcttaactgaaaaagatctgctagaagatggttttggagagcaccccttttaccactgcctggttgcagaagtgccgaaagagcactggactccggaaggacacagcattgttggttttgccatgtactattttacctatgacccgtggattggcaagttattgtatcttgaggacttcttcgtgatgagtgattatagaggctttggcataggatcagaaattctgaagaatctaagccaggttgcaatgaggtgtcgctgcagcagcatgcacttcttggtagcagaatggaatgaaccatccatcaacttctataaaagaagaggtgcttctgatctgtccagtgaagagggttggagactgttcaagatcgacaaggagtacttgctaaaaatggcaacagaggagtgaggagtgctgctgtagatgacaacctccattctattttagaataaattcccaacttctcttgctttctatgctgtttgtagtgaaataatagaatgagcacccattccaaagctttattaccagtggcgttgttgcatgtttgaaatgaggtctgtttaaagtggcaatctcagatgcagtttggagagtcagatctttctccttgaatatctttcgataaacaacaaggtggtgtgatcttaatatatttgaaaaaaacttcattctcgtgagtcatttaaatgtgtacaatgtacacactggtacttagagtttctgtttgattcttttttaataaactactctttgatttaaaaaaaaaaaaaaaaaaaaaaaaaaa

SEQ ID NO: 63 - DNA, 1117 bp, Homo sapiens
  misc_feature: Homo sapiens H3 histone, family 3A (H3F3A), mRNA NCBI NM_002107
caattgtgttcgcagccgccgccgcgccgccgtcgctctccaacgccagcgccgcctctcgctcgccgagctccagccgaaggagaaggggggtaagtaaggaggtctctgtaccatggctcgtacaaagcagactgcccgcaaatcgaccggtggtaaagcacccaggaagcaactggctacaaaagccgctcgcaagagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctcaaaaaaaaaaaaaaaaatttctcttcttcctgttattggtagttctgaacgttagatattttttttccatggggtcaaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttttttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaactataaaaaaaaaaaaaaaaaaaaaaaaaaaaaaaaaaaaaaaaaaaaaaaaaaaaaaaaaaaaaaaaaaaaa

SEQ ID NO: 64 - DNA, 1665 bp, Homo sapiens
  misc_feature: Homo sapiens interleukin 8 (IL8), mRNA NM_000584
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaa

SEQ ID NO: 65 - DNA, 366 bp, Prevotella melaninogenica
  misc_feature: Prevotella melaninogenica partial 16S rRNA gene NCBI AJ555137
ggctcaggatgattttctagctacaggcttaacacatgcaagtcgaggggaaacggcattgagtgcttgcactctttggacgtcgaccggcgcacgggtgagtaacgcgtatccaaccttcccattactgtgggataacctgccgaaaggcagactaatacctgcatagtcttcgatgacggcatcagatttgaagtaaagatttatcggtaatggatggggatgcgtctgattagcttgttggcggggtaacggcccaccaaggctacgatcagtaggggttctgagaggaaggtcccccacattggaactgagacacggcccaaactcctacgggaggcagcaatcgaattcccgcttgccaca

SEQ ID NO: 66 - DNA, 1454 bp, Streptococcus mitis
  misc_feature: Streptococcus mitis partial 16S rRNA gene, strain B6 NCBI AJ617805
cgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtttgatagatga

SEQ ID NO: 67 - DNA, 1480 bp, Capnocytophaga gingivalis
  misc_feature: Capnocytophaga gingivalis 16S ribosomal RNA gene  NCBI AF543295
agagtttgatcctggctcagatgaacgctagcggcaggcctaacacatgcaagtcgagggagaagccctttcgggggcagaaaccggcgcacgggtgcgtaacgcgtatgcaacctacctttcacagggggatagcccgaagaaatttggattaataccccataatattattggatggcatcatttgataattaaaactgcggtggtgaaagatgggcatgcgtcctattagctagttggagtggtaacggcaccccaaggctacgataggtaggggtcctgagagggagatcccccacactggtactgagacagggaccagacccctacgggaggcagcagtgaggaatattggtcaatggtcggaagactgaaccagccatgccgcgtgcaggaagaatgccttatgggttgtaaactgcttttatatgggaagaataaggtgtacgtgtacattgatgacggtaccatatgaataagcatcggctaactccgtgccagcagccgcggtaatacggaggatgcgagcgttattcggaatcattgggtttaaagggtctgtaggcgggctattaagtcaggggtgaaaggtttcagcttaactgagaaattgcctttgatactggtagtcttgaatatctgtgaagttcttggaatgtgtagtgtagcggtgaaatgcttagatattacacagaacaccgattgcgaaggcaggggactaacagacaattgacgctgagagacgaaagcgtggggagcgaacaggaattagatcccctggtagtccacgcctgtaaacgatggatactagctgttgggcgcaggctgagttggcttaagcgaaagtgataagtatccaaccttggggaagtacgcacgcaagtgtgaaactcaaaggaattgacgggggcccgcacaagcggtggagcatgtggtttaattcgatgatacgcgaggaaccttaccaaggtttaaatggagactgacaggtgtagagatacgcccttcttcggacagttttcaaggtgctgcatggttgtcgtcagctcgtgccgtgaggtgtcaggttaagtcctataacgagcgcaacccctattgttagttaccagcaagtaaagttggggactctagcaagactgccggtgtaaaccgtgaggaaggtggggatgacgtcaaatcatcacggcccttacatcttgggctacacacgtgctacaatggtcgttacagagagcagccactgcgcgagcaggagcgaatctataaagacggtcacagttcggatcggagtctgcaactcgactccgtgaagctggaatcgctagtaatcggatatcagccatgatccggtgaatacgttcccgggccttgtacacaccgcccgtcaagccatggaagctgggagtacctgaagtcggtcaccgcaaggagctgcctagggtaaaaccagtgactggggctaagtcgtaacaaggtaacc

SEQ ID NO: 68 - DNA, 838 bp, Micrococcus luteus
  misc_feature: Micrococcus luteus partial 16S rRNA gene, isolate A24 NCBI AM285006
aggatgaacgctggcggcgtgcttacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcgcaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggttttccgcgccgcagctaacgcattaagtgccccgccct

SEQ ID NO: 69 - DNA, 297 bp, Human papillomavirus type 16
  misc_feature: Human papillomavirus type 16 strain P209 E7 protein (E7) gene,complete cds NCBI EF422141
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 70 - DNA, 477 bp, Human papillomavirus type 18
  misc_feature: Human papillomavirus type 18 strain P629 E6 protein (E7) gene,complete cds NCBI EF422111
atggcgcgctttgaggatccaacacggcgaccctacaagctacctgatctgtgcacggaactgaacacttcactgcaagacatagaaataacctgtgtatattgcaagacagtattggaacttacagaggtatttgaatttgcattcaaagatttatttgtagtgtatagagacagtataccgcatgctgcatgccataaatgtatagatttttattctagaattagagaattaagacattattcagactctgtgtatggagacacattagaaaaactaactaacactgggttatacaatttattaataaggtgcctgcggtgccagaaaccgttgaatccagcagaaaaacttagacaccttaatgaaaaacgacgattccacaaaatagctgggcactatagaggccagtgccattcgtgctgcaaccgagcacgacaggagagactccaacgacgcagagaaacacaagtataa